(19) United States
(12) Patent Application Publication
(10) Pub. No.: US 2024/0207599 A1
(43) Pub. Date: Jun. 27, 2024

(54) NEXT GENERATION TOTAL ARTIFICIAL HEART SYSTEM

(71) Applicant: SYNCARDIA SYSTEMS, LLC, Tucson, AZ (US)

(72) Inventor: Matthew Scott Schuster, Tucson, AZ (US)

(21) Appl. No.: 18/600,056

(22) Filed: Mar. 8, 2024

Related U.S. Application Data
(63) Continuation-in-part of application No. 18/432,598, filed on Feb. 5, 2024.
Application No. 18/432,598 is a continuation of application No. 17/779,058, filed on May 23, 2022, now Pat. No. 11,918,798.
(60) Provisional application No. 62/938,733, filed on Nov. 21, 2019.

(51) Int. Cl. A61M 60/196

(52) U.S. Cl. CPC A61M 60/196

(57) ABSTRACT

A total artificial heart system includes at least one artificial ventricle coupled to (or capable of being coupled to) a chamber or a vessel of a human heart and at least one drive system coupled to the artificial ventricle. The drive system contains at least one implanted in a chest cavity electric motor. The drive system causes the artificial ventricle to contract and/or expand in a specific fashion dependent on a value inversely proportional to a rotational speed of a rotorcam and/or a height of the cam follower of the heart system.

### CROSS-REFERENCE TO RELATED APPLICATIONS

This US Patent Application is a continuation-in-part from the U.S. patent application Ser. No. 18/432,598, which is a continuation from the U.S. patent application Ser. No. 17/779,058 filed on May 23, 2022 and now granted as U.S. Pat. No. 11,918,798, which is a national phase of the International Patent Application No. PCT/US2020/060785 filed on Nov. 16, 2020 and now published as WO 2021/101848, which in turn claims priority from and benefit of the U.S. Provisional Patent Application No. 62/938,733 filed on Nov. 21, 2019. The disclosure of each of the above-identified patent documents is incorporated herein by reference.

### TECHNICAL FIELD

This patent application relates to an electric-moto driven artificial heart apparatus or system and methodologies for actuating such apparatus.

### RELATED ART

The heart is the muscle that drives the cardiovascular system in humans and other living beings. Acting as a pump, the heart moves blood throughout the body to provide oxygen, nutrients, hormones, and to remove waste products. The blood follows two separate pathways in the human body, the so-called pulmonary circulatory circuit and systemic circulatory circuit. In the pulmonary circuit, the heart pumps blood first to the lungs to release carbon dioxide and bind oxygen, and then back to the heart. Thus, oxygenated blood is constantly being supplied to the heart. In the systemic circuit, the longer of the two, the heart pumps oxygenated blood through the rest of the body to supply oxygen and remove carbon dioxide, the byproduct of metabolic functions carried out throughout the body. The heart supplies blood to the two circuits with pulses generated by the orderly muscular contraction of its walls.

In order to keep blood moving through these two separate circulatory circuits, the human heart has four distinct chambers that work in pairs. As illustrated in FIG. 1, the heart 10 includes four chambers: a right atrium 12, a right ventricle 14, a left atrium 16, and a left ventricle 18. One pair of chambers, the right ventricle and left atrium, is connected directly to the pulmonary circuit. In it, de-oxygenated blood from the body is pumped from the right ventricle 14 to the lungs, where it is enriched with oxygen, and then pumped back to the left atrium 16.

In the systemic circuit, the other pair of chambers pumps the oxygenated blood through body organs, tissues, and bones. The blood moves from the left atrium 16 (to where it is delivered from the lungs) to the left ventricle 18, which in turn pumps the blood throughout the body and all the way back to the right atrium 12. The blood then moves to the right ventricle 14—and the cycle is repeated. In each circuit, the blood enters the heart through an atrium and leaves the heart through a ventricle.

Thus, the ventricles 14, 18 are essentially two separate pumps that work together to move the blood through the two circulatory circuits. Four check valves control the flow of blood within the heart and prevent flow in the wrong direction. A tricuspid valve 20 controls the blood flowing from the right atrium 12 into the right ventricle 14. Similarly, a bicuspid valve 22 controls the blood flowing from the left atrium 16 into the left ventricle 18. Two semilunar valves (pulmonary semilunar valve 24 and aortic semilunar valve 26) control the blood flow leaving the heart toward the pulmonary circuit and the systemic circuit, respectively. Thus, in each complete cycle, the blood is pumped by the right ventricle 14 through the pulmonary semilunar valve 24 to the lungs and back to the left atrium 16. The blood then flows through the bicuspid valve 22 to the left ventricle 18, which in turn pumps it through the aortic semilunar valve 26 throughout the body and back to the right atrium 12. Finally, the blood flows back to the right ventricle 14 through the tricuspid valve 20 and the cycle is repeated.

When the heart muscle squeezes each ventricle, the ventricle acts as a pump that exerts pressure on the blood, thereby pushing it out of the heart and through the body. The blood pressure, an indicator of heart function, is measured when the heart muscle contracts as well as when it relaxes. The so-called systolic pressure is the maximum pressure exerted by the blood on the arterial walls when the left ventricle of the heart contracts and forces blood through the arteries in the systemic circulatory circuit. The so-called diastolic pressure is the lowest pressure on the blood vessel walls when the left ventricle relaxes and refills with blood. Healthy blood pressure is considered to be about 120 millimeters of mercury systolic and 80 millimeters of mercury diastolic (usually presented and/or identified as 120/80).

Inasmuch as the function of the circulatory system is to service the biological needs of all body tissues (i.e., to transport nutrients to the tissues, to transport waste products away, to distribute hormones from one part of the body to another, and, in general, to maintain an appropriate environment for optimal function and survival of tissue cells), the rate at which blood is circulated by the heart is a critical aspect of its function. The heart has a built-in mechanism (the so-called Frank-Starling mechanism) that allows it to pump automatically whatever amount of blood flows into it. Such cardiac output in a healthy human body may vary from about 4 to about 15 liters per minute (LPM), according to the activity being undertaken by the person, at a heart rate that can vary from about 50 to about 180 beats per minute.

Several artificial devices have been developed over the years to supplement or replace the function of a failing heart in patients. These include devices developed by companies as well as research institutions such as the Berlin Heart Institute, the Pennsylvania State University, the University of Utah, the Cleveland Clinic Foundation, the University of Perkinje (in Bruno, Czechoslovakia), the University of Tokyo, the Thoratec Corporation, Abiomed Inc., Novacor, and Symbion Inc. Typically, these artificial devices consist of pumps that aim at duplicating the required pumping functions of the left and right human ventricles. One method of actuation for these pumps has been through the pneumatic action of an external mechanism. See, for example, U.S. Pat. Nos. 4,611,578 and 5,766,207. Periodic pulses of compressed air drive the pumps at the desired pressure and rate of cardiac output. A moderate vacuum may be applied between pulses to allow more rapid refilling of the ventricles with blood flowing from the respective atrium.

The pneumatic drivers used to date for driving all artificial hearts have been cumbersome and inadequate for affording patients any degree of independent mobility. Such drivers employ compressors, vacuum pumps, and air tanks coupled to electrically actuated valves, all of which amounts to a large and heavy apparatus that can only be transported on wheels and with considerable effort. Therefore, many attempts have been made during the last two decades to produce a portable driver for these devices. However, because of the complexity of the required functionality and the hardware necessary to produce it, pneumatic heart drivers continue to be bulky, require frequent maintenance, and often provide air pulses that do not match the performance of the larger drivers they are meant to replace. Even at the approximate weight of 15 pounds and size of about 0.7 cubic feet achieved so far, pneumatic drivers remain unwieldy and substantially not portable for a patient who is kept alive by an artificial heart.

In essence, a portable driver needs to be reliable, durable, easy to use, and sufficiently simple in design to be affordable. Unfortunately, each of these requirements contributes to the complexity of the design, which in turn was realized in devices that are not sufficiently small and light-weight to be manageable in the hands of a patient. Furthermore, it is essential that the pneumatic driver be able to provide the correct pressure balance between the left and right ventricles of the artificial heart to ensure the proper operating pressure to the pulmonary and systemic circuits regardless of the speed of operation. Typically, such consideration leads to the requirement for the driver to be able to operate so as to maintain, on average, a right atrial pressure of about 9 mmHg, a mean pulmonary artery pressure of about 35 mmHg, a left atrial pressure of about 10 mmHg, and a mean aortic pressure of about 95 mmHg.

This need to provide different operating pressures to the right and left lower chambers (the right and left ventricles) of the artificial-heart device has not been met thus far with a simple design suitable for a portable driver. For example, the blood pump described in U.S. Pat. No. 4,611,578 includes a configuration in which two reciprocating pistons in a common cylinder may be operated alternatively to provide redundancy or independently to actuate two separate pneumatically driven blood pumps. While the question of different operating pressure is not addressed in the patent, the disclosure presents a sophisticated control system that arguably could be used to provide the correct operating pressure to each lower chamber of the artificial heart. However, the complex and multi-component structure of the device necessarily requires a relatively heavy and large apparatus, though described as portable: the commercially available module weighs about 25 pounds and is approximately 0.6 cubic feet in volume.

U.S. Pat. No. 5,766,207 describes another portable pneumatic driver for ventricular assist devices that could also be adapted for an artificial heart. The single pump as discussed could be used to drive both ventricles of an artificial heart, but only at the same pressure and volume rate. Thus, the disclosed device, even if modified to meet the other requirements of a portable artificial-heart driver, would not be suitable as an alternative to the stationary modules currently in use.

In one or more lighter options, a system of related art may include a portable case weighing about fifteen (15) pounds, and including one or more compressors acting as drivers for the pneumatic system. Because such arrangement involves pushing pulses of air through tubes into and out of the artificial heart, high fluid and/or backpressure losses occur resulting in limited battery life (i.e., due to the high amperage or current drawn by the compressors). For example, in one or more systems, the device runs for approximately 2 hours before batteries need to be replaced or recharged. In addition, the patient is required to carry the system, which limits the activities the patient may undertake due to at least one hand or arm having to carry the system. Such system may also include hundreds of individual parts, which may have an impact on the reliability of the system. In addition, in order to approximate the displacement of blood, the volumetric airflow through such system (which itself presents uncertainty of operation) needs to be determined.

Other systems of related art use impellers (or rotational pumping devices) to directly pressurize and pump the blood. However, impactions and/or contact between the impeller and the blood create high sheer stresses, which can damage the blood.

Therefore, the current options available to patients do not meet the desired characteristics, which include providing a mobile, highly reliable, light-weight, manageably-sized, and portable system for a patient in need of a heart transplant.

### SUMMARY OF THE INVENTION

Embodiments of the invention provide a total artificial heart system that includes: an artificial ventricle (that is configured to be implanted in a chest cavity of a patient and to be coupled to at least one a blood vessel extending out of or arising from a heart, and a chamber of a heart) and a drive system (that has an electric motor including a stator assembly, a rotocam coupled to the stator assembly, and a cam follower coupled to the rotorcam). Here, the drive system is configured to be implanted in a chest cavity of a patient, to be coupled to the artificial ventricle, and configured such that a magnitude of a change of a volume of the artificial ventricle is substantially inversely proportional to a rotational speed of the rotorcam. In at least one implementation, the system includes a diaphragm a diaphragm configured as a boundary between the artificial ventricle and the drive system (where on a ventricle side, the at least one diaphragm is configured to contact human blood, and on a side of the drive system the at least one diaphragm is configured to contact a gas). Optionally, the drive system may be structured to change a volume of the artificial ventricle by changing a height of the cam follower. Alternatively or in addition, and in at least one implementation, the system is characterized by at least one of the following features: (i) the system includes at least one spring coupling the cam follower to the stator assembly, where the stator assembly comprises a shaped housing with a first shaped cross-section and where the at least one cam follower comprises a shaft with a second shaped-cross section matching the first shaped cross-section of the shaped housing; (ii) the rotorcam includes a center bore concentric about a centerline of the rotorcam and a ramp radially disposed around the center bore; (iii) the rotorcam includes at least one of neodymium iron boron (Nd—Fe—B), iron, cobalt, samarium cobalt (Sm—Co), aluminum, alnico, bonded Nd—Fe—B, magnetite, ceramic (hard ferrite), ferrite, gadolinium, one or more rare earth elements, strontium, barium, and iron (III) oxide; (iv) the system further contains at least one proximity sensor disposed within at least one of the stator assembly and the cam follower, where the at least one proximity sensor is configured to detect a relative position of the cam follower and the stator assembly with respect to one another; (v) the system further includes at least one pressure sensor disposed on at least one of the stator assembly and the rotorcam; (vi) the rotorcam contains a rotocam bore, the stator assembly has a cylindrical housing, and the cylindrical housing is disposed within the rotorcam bore such that the rotorcam extends circumferentially around, and radially outward of, the cylindrical housing; (vii) the stator assembly contains a stator assembly cylindrical housing, where the rotorcam is disposed about the stator assembly cylindrical housing, and wherein the system is configured to have a rotation of the rotorcam about the stator assembly cylindrical housing to cause a change in a height of the cam follower. Optionally, in such a case, the rotorcam may be structured to include the ramp radially disposed around the center bore, while the system may be configured to satisfy at least one of the following conditions: (a) the ramp includes at least one drop-off, an inclined portion, and a flat portion disposed between the inclined portion and the drop-off; and (b) the at least one cam follower includes at least one tooth extending toward the rotorcam and interfacing with a portion of the ramp. Alternatively or in addition, and at least in one embodiment, the system may be configured to contain at least one spring while the stator assembly has the shaped housing with the first shaped cross-section and while the cam follower has the shaft with the second shaped-cross section (here, each of the first and second shaped cross-sections may include at least one of a triangular cross-section, a square cross-section, an elliptical cross-section, a pentagonal cross-section, a hexagonal cross-section, and a rectangular cross-section). Alternatively or in addition, the system maybe structured to include the at least one spring while the stator assembly has the shaped housing with the first shaped cross-section and while the cam follower has the shaft with the second shaped-cross section (here, the shaft may be disposed within the housing and is configured to move longitudinally within the housing). Alternatively or in addition, the stator assembly may have the shaped housing that includes at least one damper disposed therewithin; and/or, when the rotorcam contains the rotocam bore and when the stator assembly has the cylindrical housing disposed within the rotorcam bore such that the rotorcam extends circumferentially around (and radially outward of) the cylindrical housing, the system may be additionally structured to include a retaining rim disposed around a top of the cylindrical housing, such retaining rim being configured to allow the rotorcam to rotate about the cylindrical housing while preventing the rotorcam from translating longitudinally.

Embodiments of the invention additionally provide a total artificial heart system that includes (i) an artificial ventricle configured to be implanted in a chest cavity of a patient and to be coupled to at least one a blood vessel extending out of or arising from a heart, and a chamber of a heart; and (ii) a drive system having an electric motor that includes a stator assembly, a rotocam coupled to the stator assembly, and a cam follower coupled to the rotorcam. Here, the drive system is configured to be implanted in a chest cavity of a patient, to be coupled to the artificial ventricle, and configured to change a volume of the artificial ventricle by changing a height of the cam follower.

Embodiments of the invention further provide an article of manufacture that contains a cam-and-follower system. The cam-and-follower system includes (i) a monolithic rotorcam configured to operate as both a rotor and a cam of the system and to rotate about a centerline, the monolithic rotorcam comprising at least two angled ramps circumferentially extending around a first axis of the monolithic rotorcam, an operational height of the cam of the system defined by a height of an angled ramp of the at least two angled ramps; and (ii) a cam follower that has a second axis of the cam follower and a dome having a center of curvature of the dome on the centerline. Here, (a) the at least two angled ramps include an outer ramp having an outer ramp radius; and at inner ramp having an inner ramp radius that is smaller than the outer ramp radius; and/or (b) the cam follower includes at least two teeth extending from the dome toward the monolithic rotorcam, where each of the at least two teeth is dimensioned to contact a corresponding angled ramp of the at least two angled ramps when the cam-and-follower system is assembled. At least in one specific implementation of the article, the cam follower may be structured to be devoid of such constituent components that are configured to move with respect to one another; and/or the cam follower may be configured as a monolithic component; and/or a spatial profile of a tooth of the at least two teeth may be made not congruent with a spatial profile of the corresponding angled ramp of the at least two angled ramps. In at least one specific implementation, the article of manufacture may be structured such that each of the present angled ramps of the monolithic rotorcam includes (i) a corresponding inclined portion having a first ramp height at a first ends thereof and a second ramp height at a second end thereof, (ii) a corresponding drop-off portion sharing the second end with the inclined portion and having a drop-off surface that is substantially transverse to an upper surface of the inclined portion, and (iii) a corresponding flat portion disposed between the inclined portion and the drop-off portion and having an upper surface of the flat portion that substantially seamlessly merging with the upper surface of the inclined portion and the drop-off surface. Here, a height of the ramp may be defined to remain substantially equal to the first ramp height along the flat portion and the second ramp height is larger than the first ramp height.

Embodiments of the invention further include a method that (with the use of an embodiment of the article of manufacture itemized above that has been implanted in a chest cavity of a patient in contact with a flexible diaphragm of an artificial ventricle that has been implanted in the chest cavity) effectuates (i) rotating a monolithic rotocam, configured to operate as both a rotor and a cam of a cam-and-follower system of said article, about a centerline; and (ii) changing a magnitude of expansion of the artificial ventricle inversely proportionally to a rotations speed of the rotorcam, and/or changing a volume of the artificial ventricle by changing a height of the cam follower with respect to a base on the rotorcam.

Embodiments of the invention additionally provide an article of manufacture that includes a cam and follower system. Such cam and follower system contains: a monolithic rotorcam configured to operate as both a rotor and a cam of the system and to rotate about a centerline, the monolithic rotorcam having a first axis and comprising an angled cam ramp circumferentially extending around the first axis and including at least one contoured cam lobe extending along the first axis (where the angled cam ramp has a first edge surface), and a cam follower having a second axis and a dome and comprising an angled cam follower ramp circumferentially extending around a perimeter of the dome and including at least one contoured cam follower lobe (the angled cam follower ramp having a second edge surface dimensioned to be substantially congruent with the first end surface). Here, the cam follower is configured to be mechanically coupled to the monolithic rotorcam when the cam-and-follower system is assembled such that in an axial separation between the first and second edge surfaces is substantially zero. Optionally, the cam follower may be structured to be devoid of constituent components configured to move with respect to one another and/or be structured to be monolithic. Alternatively or in addition, in a given embodiment of the article of manufacture, a height of each of the angled cam ramp and the angled cam follower ramp may be defined to be monotonically and uninterruptingly changing around a corresponding circumference. Optionally, the cam follower may have a shaft monolithically extending from the dome along a radius of a curvature of a convex surface of the dome, while the rotorcam is made monolithic and includes an axial center bore and dimensioned to accommodate the shaft therethrough. Optionally—and substantially in every implementation—the article may additionally include a stator assembly having a third axis, a base plate substantially perpendicular to the third axis, and a base housing extending from the base plate along the third axis and defining a hollow that is substantially centered at the third axis (here, when the cam follower has a shaft monolithically extending from the dome along a radius of a curvature of a convex surface of the dome, an inner perimeter of the hollow is preferably structured to be substantially geometrically matching an outer perimeter of a shaft of the cam follower that extends along the second axis way from an outer surface of the dome). The inner perimeter of the hollow may be structured as a polygon, while the stator assembly may include multiple groups of wound coil wires disposed about the inner perimeter and configured to be electrically activated sequentially around the third axis. Alternatively or in addition, and substantially in every implementation, the article may be further configured to include a drive system having an electric motor that includes (when the rotorcam and the stator assembly and the cam follower are assembled to have the first axis and the second axis and the third axis substantially coincide) (i) the rotorcam sandwiched between the stator assembly and the cam follower such that the shaft passes through a center bore of the rotorcam and is received and accommodated within the hollow of the stator assembly without an ability to rotate about the second axis within the hollow; and/or (ii) a first of the at least two teeth in contact with a ramp surface of one of the inner ramp and the outer ramp and a second of the at least two teeth be in contact with a ramp surface of the other of the inner ramp and an outer ramp. In the latter case, an axial length of the article of manufacture may be defined by an axial separation between a surface of the base plate and a surface of the dome. In at least one implementation of the article, the cam-and-rotor system may be configured to be implanted in a chest cavity of a patient, while the article of manufacture further includes an artificial ventricle configured to be implanted in a chest cavity of a patient and coupled to the cam-and-follower system and be further configured (a) to change a volume of the artificial ventricle substantially inversely proportionally to a rotational speed of the rotocam, and/or (b) to change a volume of the artificial ventricle by changing a separation between the dome of the cam follower and the circular base of the stator assembly.

### BRIEF DESCRIPTION OF THE DRAWINGS

The invention will be more fully understood by referring to the following Detailed Description of Specific Embodiments in conjunction with the not-to scale Drawings, of which:

FIG. 1 illustrates schematically a human heart in a cross-sectional front view;

FIG. 2 presents a front view of a total artificial heart system, structured according to the idea of the present invention;

FIG. 3 illustrates a schematic an implanted total artificial heart system, operationally coupled with at least a portion of the human heart, according to the idea of the present invention;

FIG. 4 shows schematically a human with the implanted total artificial heart system;

FIG. 5 illustrates an embodiment of the total artificial heart drive system in a perspective view;

FIG. 6 illustrates an embodiment of a drive system stator assembly base, configured according to principles of the present invention;

FIG. 7 provides a perspective view of an embodiment of a drive system rotorcam, configured for use with an embodiment of the system of the invention;

FIG. 8 illustrates, in a perspective view, an embodiment of a drive system cam follower of the total artificial heart system structured according to the idea of the invention;

FIG. 9 presents, in a perspective view, the assembled version of the drive system shown in FIG. 5;

FIG. 10 illustrates another a perspective view of the assembled drive system;

FIGS. 11, 12, 13, 14, and 16 provide various perspective views the an assembled drive system cooperated with an artificial ventricle

FIG. 16 is a perspective view of an embodiment of the drive system stator assembly;

FIG. 17 shows, in a perspective view, an embodiment of a drive system stator assembly;

FIG. 18 is a top view of an embodiment of the drive system stator assembly;

FIG. 19 is a perspective view of an embodiment of a drive system dual ramp rotorcam of the system of the invention;

FIG. 20 illustrates, in a perspective view, an embodiment of a drive system dual cam-follower;

FIG. 21 illustrates a perspective view of a disassembled drive system, according to aspects of the present embodiments;

FIG. 22 illustrates a perspective view of an assembled drive system, according to aspects of the present embodiments;

FIG. 23 illustrates a perspective view of an assembled emperor crown drive system, according to aspects of the present embodiments;

FIG. 24 illustrates a perspective view of a control module, according to aspects of the present embodiments;

FIG. 25 illustrates a perspective view of a battery charging system, according to aspects of the present embodiments;

FIG. 26 illustrates a front view of a mobile device application, according to aspects of the present embodiments;

FIG. 27 illustrates a front view of an implanted total artificial heart system, according to aspects of the present embodiments;

FIG. 28 illustrates a schematic of an electrical current versus volume of blood characteristic, according to aspects of the present embodiments;

FIG. 29 illustrates a schematic of volume of air versus pressure of air characteristic, according to aspects of the present embodiments;

FIG. 30 illustrates a schematic of volume of blood versus pressure of blood characteristic, according to aspects of the present embodiments;

FIG. 31 illustrates a schematic of volume of air versus pressure of air characteristic, according to aspects of the present embodiments;

FIG. 32 illustrates a schematic of ramp height versus rotor angle characteristic, according to aspects of the present embodiments;

FIG. 33 illustrates a schematic of both follower height and rotor velocity versus rotor angle characteristic, according to aspects of the present embodiments;

FIG. 34 illustrates a schematic of both follower height and rotor velocity versus rotor angle characteristic, according to aspects of the present embodiments;

FIG. 35 illustrates a schematic of a method of implanting a total artificial heart, according to aspects of the present embodiments;

FIG. 36 illustrates a schematic of a method of calibrating a total artificial heart, according to aspects of the present embodiments; and

FIG. 37 illustrates a schematic of a method of operating a total artificial heart, in accordance with aspects of the present disclosed embodiments.

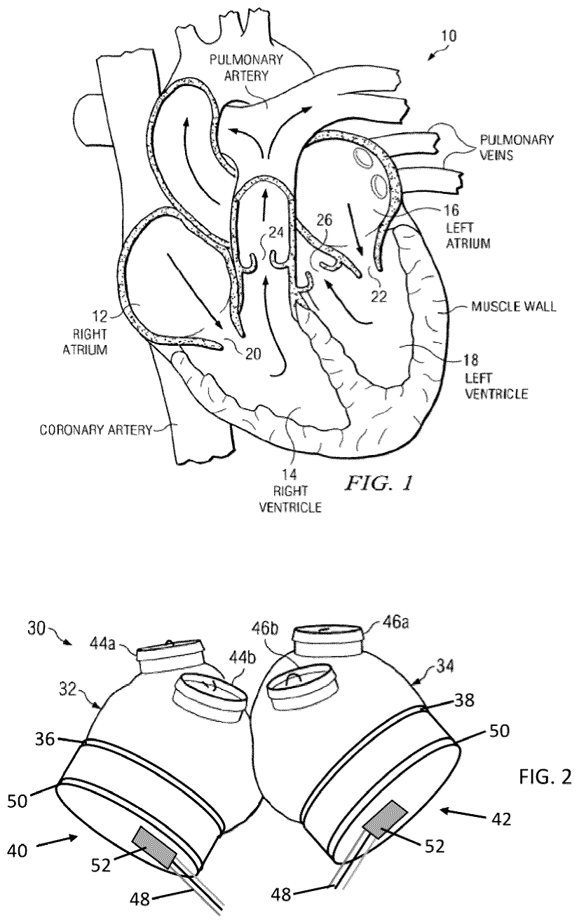

Reference will now be made in detail to the present disclosed embodiments, one or more examples of which are illustrated in the accompanying drawings. The detailed description uses numerical and/or letter designations to refer to features in the drawings. Like or similar designations in the drawings and description have been used to refer to like or similar parts of the present embodiments.

### TOTAL ARTIFICIAL HEART SYSTEM

According to the idea of the invention and designed to operate much the same way as a human heart, an embodiment of the total artificial heart (TAH) system is configured to replace the two active chambers (i.e., the ventricles) of the human heart with corresponding artificial components. As illustrated in FIG. 2, such TAH system 30 includes two separate chambers or ventricles 32, 34 that replace the right and left ventricles of the human heart, respectively. Each artificial chamber/ventricle 32, 34 is equipped with a respective flexible diaphragm (36 and 38 in the right and left chamber, respectively) that has an air contact side and a blood contact side. Each diaphragm 36, 38 may be designed to assume, in at least one orientation, a shape substantially congruent with that of a hemisphere. In practice, the TAH system 30 is made biologically compatible and is implanted by connecting the top of the right chamber 32 to an identified portion of the right atrium 12 and the top of the left chamber 34 to the identified portion of the left atrium 16. The bottom of each of artificial ventricles is complemented and equipped with a corresponding drive system (40 and 42, for the right and left artificial ventricles, respectively) that is also in practice embedded in the patient's body. Each drive system includes electrical members (such as wires) 48 extending out of the patient's body. Each drive system 40, 42 generally uses a gas (for example, inert gas; or, in a specific embodiment—air) to push against the respective diaphragm 36, 38 of the artificial left and right ventricles 32, 34, 38, which discharges blood from the respective artificial ventricle 32, 34, thereby simulating and performing the function of a ventricle of the human heart. As such, each diaphragm 36, 38 defines a transition or boundary between the respective artificial ventricle 32, 34 and the respective drive system 40, 42.

The drive systems 40, 42 may each include a base 50 (optionally dimensioned as a plate, in one case—a substantially plane-parallel plate), as well as one or more ports 52 dimensioned to allow the electrical members 48 to pass through to the base to the interior of the corresponding drive system 40, 42. The TAH system 30 may also include artificial valves 44a (configured to operate as tricuspid valves), 46a (bicuspid), 44b (pulmonary), and 46b (aortic), which in operation control the flow of blood from the respective atrium into a corresponding artificial ventricle 32, 34, and out to the circulatory systems, respectively.

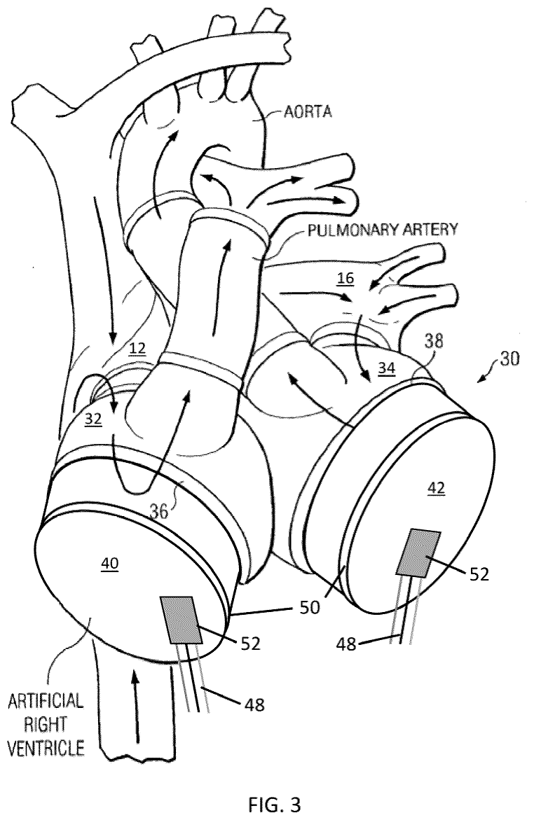

FIG. 3 presents, in a perspective view, the implanted TAH system 30 fluidly and physically connecting the top of the right artificial chamber 32 to the right atrium 12 and the top of the left artificial chamber 34 to the left atrium 16. The bottom of each artificial chamber is provided and equipped with a corresponding drive system (40 and 42, in the right and left artificial chamber, respectively) that is as well embedded in the patient's body and includes electrical members (wires as shown) that extend out of the patient's body to deliver electrical power to corresponding drive systems. As each drive system 40, 42 uses the driving fluid of choice (generally, the gas of choice, in one example—air) to push against respective flexible diaphragm 36, 38 the respective artificial left and right ventricles 32, 34, the volume of the corresponding ventricle (on the blood-side of the diaphragm) limited by the diaphragm and the inner surface of the ventricle's shell is reduced or contracted, and blood is discharged from each chamber 32, 34, thereby pumping blood out of the heart during a systole or ejection phase. When the driving fluid (for example, pressurized air) is caused to be outflown away from, removed from the chosen diaphragm (in a part of the operational cycle known as diastole or the filling phase), blood can enter the expanding volume of the ventricle from the connected atrium. The rate at which blood enters the ventricle depends on the difference between the atrial pressure and the pressure on the air-side of the diaphragm. To increase the rate of filling of the volume of the ventricle (referred to herein as the filling rate), a slight vacuum of about 10 mmHg on average may be established within the drive system 40, 42 on the air-side of the diaphragm 36, 38 during diastole. The pressure is higher during systole.

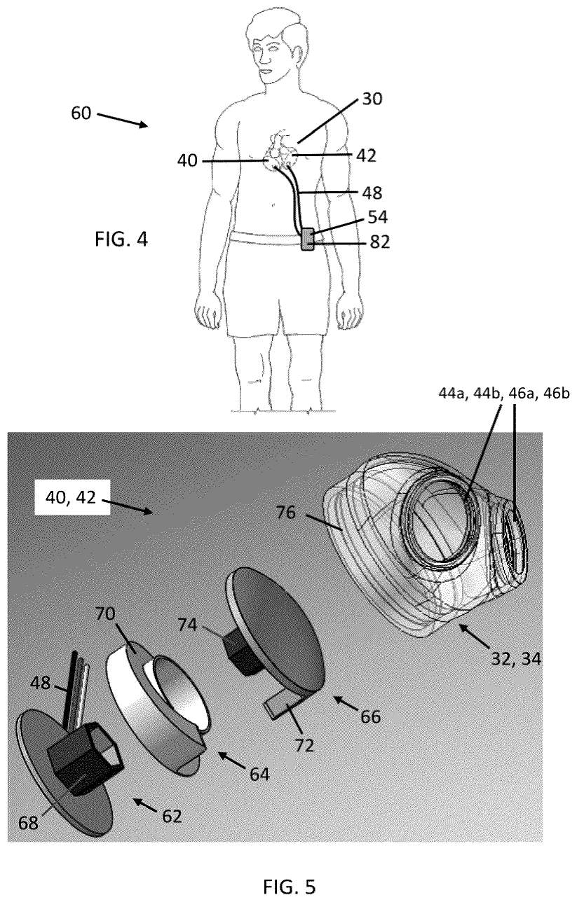

FIG. 4 illustrates, in a front view, the total artificial heart system 30 implanted within the chest cavity of the patient 60. the TAH system 30 is shown to include right and left drive systems 40, 42 also implanted within the chest cavity of a patient. Each drive system 40, 42 may be connected to a mobile power supply 54 via one or more wires 48. The mobile power supply 54 may include a local interface (not shown) configured to control the function of the total artificial heart system, as well as battery ports and local control keys or buttons. In operation, the local power supply 54 is, at least in one specific case, worn on a belt or strapped around the chest, shoulder, or waist of the patient, or carried in a pocket, such that the patient is enabled to move about freely without having to carry a mobile compression unit within one or both hands (as is customary when using some of the TAH systems of related art). In addition, the mobile power supply 54 is configured to weigh generally less than or about three (3) pounds, or from about two (2) pounds to about three (3) pounds, or from about one (1) pound to about two (2) pounds, or less than about one (1) pound—depending on the specifics of a particular implementation. In related specific embodiments, the overall weight of the mobile power supply 54 may be from about four (4) ounces to about sixteen (16) ounces, or from about six (6) ounces to about fourteen (14) ounces, or from about eight (8) ounces to about twelve (12) ounces.

### Drive System

FIG. 5 presents a perspective exploded view of one embodiment of a (disassembled) drive system 40, 42 of the total artificial heart system 30, structured according to the idea of the invention. As shown in FIG. 5, the drive system 40, 42 would be coupled (when assembled) to the corresponding right or left artificial ventricle 32, 34. In one implementation, as shown, the drive system 40, 42 includes a drive system stator assembly 62, a drive system rotorcam 64, and a drive system cam follower 66.

A will be understood from the discussion below, the cam-and-follower system of the drive system 40, 42 includes a rotorcam that is configured to operate both as a rotor and as a cam, and a cam follower (both of which two components is monolithic and thus devoid of portions moving with respect to one another). In particular, the cam follower is devoid of (does not include) a roller component to minimize mechanical motion present in the cam-and-follower system and to avoid a need to keep reducing friction and wear and tear in a contact between two surfaces of the follower (which need necessarily persists when a roller is present, as the skilled person readily appreciates).

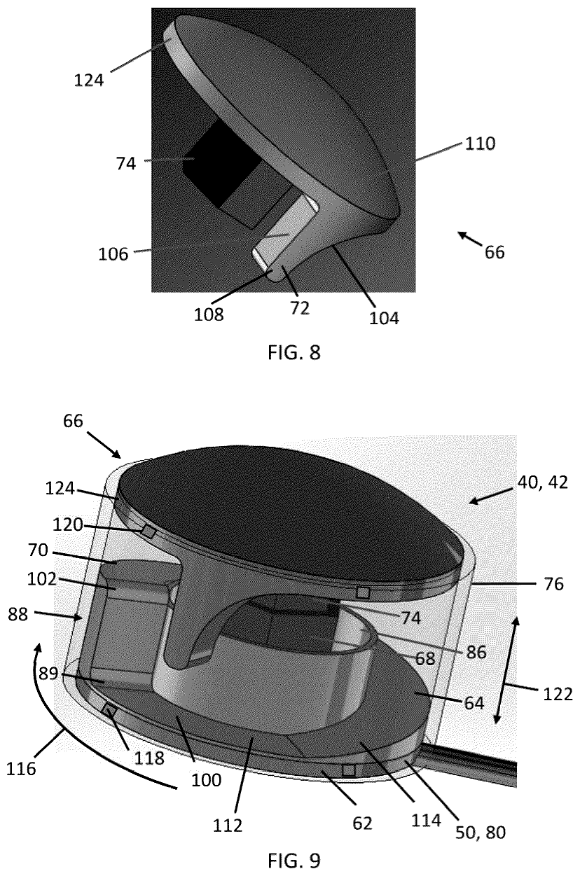

The stator assembly 62 may be electrically coupled to the electrically conducting members (such as wires) 48 to supply electrical power is supplied to the stator assembly 62. In at least one specific case, the stator assembly is configured to act and acts, in operation, as a base of an electric motor, for example a brushless director current (BLDC) motor. The rotorcam 64 is dimensioned to slide onto the stator assembly 62 around a base housing 68 (which is this embodiment has a hexagonal shape). The rotorcam 64 is configured to act—and acts, in operation—both as a rotor (as part of the electric motor) and as a cam (as part of a cam-and-follower system 40, 42). As such, the rotorcam 64 may be structured to optionally include one or more permanent magnets and/or may be composed of a solitary permanent magnetic material. In addition, the rotorcam 64 may include at least one angled ramp 70 for use in the cam-and-follower system. The specific implementation of the follower 66 shown in FIGS. 5 and 8 includes a longitudinally extending shaft 74, a shaft-capping element shaped as a dome 110 having an upper convex surface facing away from the shaft 74, and at least one tooth 72 extending substantially longitudinally (that is, along the same axis along which the shaft 74 is directed) from the lower surface of the dome 110 and configured to interface with the ramp (which is cam) of the rotorcam 64. The follower 66 may also include one (as shown, or more) shaft(s) 74 dimensioned to slide into and fit within the base 68 to prevent the follower 66 from rotating but, at the same time, to allow the follower 66 to slide up and down within and along the axis of the drive system 40, 42. (In the current embodiment, as shown, the shaft 74 understandably has a cross-section having a hexagonal outer perimeter while generally the outer perimeter of the cross-section of the shaft 74 is judiciously dimensioned to prevent the shaft from rotating about the axis of the shaft when the shaft is mechanically cooperated—for example, inserted in—the housing 68. For example, the matching shapes of the base housing 68 and the shaft 74 may be generally polygonal or non-circularly elliptical. In one specific case, each of the corresponding polygonal perimeters of the base housing 68 and the shaft 74 does not possess any degree of symmetry which, as shown experimentally, provides an unexpectedly high degree of stability of the shaft 74/based housing 68 mechanical coupling with one another.) Each of the right and left artificial ventricles 32, 34 may include, as shown, a lower (ventricle) housing 76 that is generally dimensioned to extend around the drive system assembly 40, 42 to allow for movement of the rotorcam 64 and follower 66 therewithin.

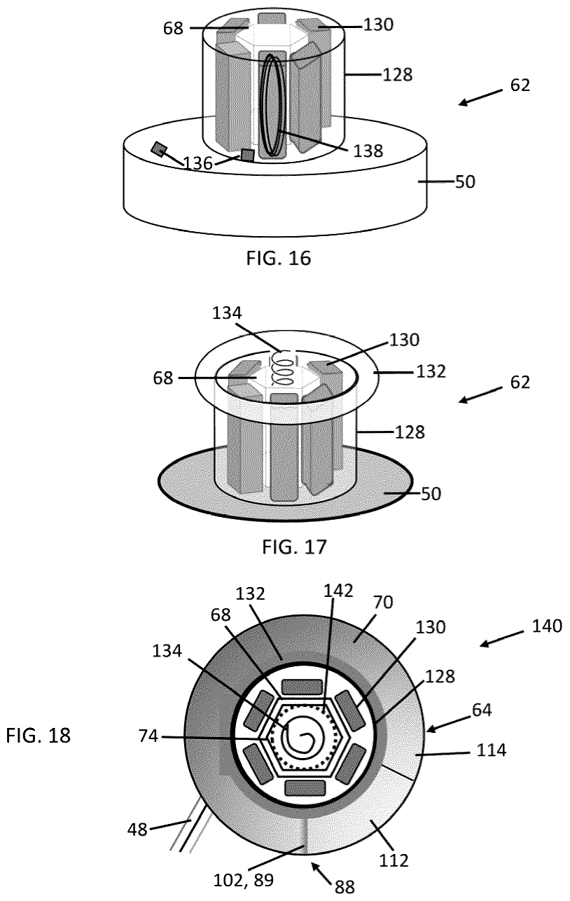

In operation, electrical charge runs through the wires 48 into the base housing 68 and eventually through one or more motor windings 130 (shown in and/or discussed below in reference to FIGS. 16-18), thereby creating an electromagnetic force (due to the presence of a permanent magnet in the system 40, 42) that causes the rotorcam 64 to rotate. A cylindrical housing 128 (shown in and/or discussed in reference to FIGS. 16, 17, and 18 below) extending from the stator assembly 62 may be dimensioned to surround both the hexagonal housing 68 and the motor windings 130 (shown in and/or discussed in reference to FIGS. 16-18), and may be disposed radially within an inner bore 78 of the rotorcam 64, thereby providing a bearing (for example, a sleeve bearing) about which the rotorcam 64 may rotate. The ramp 74 has a specific circumferentially variable height to provide for a ramp incline. As the rotorcam 64 rotates, the tip of the tooth 72 interfaces with the upper surface of the circumferentially variable ramp 70, thereby causing the follower 66 to rise and fall with the changing height of the ramp 70. A spring 134 (shown in FIG. 17) disposed within the (hexagonal) shaft 74, and coupling the follower 66 to the stator assembly 62 holds the follower 66 against the stator assembly 62, even as the ramp 70 pushes the follower 66 away from the stator assembly 62 (i.e., via the tooth 72). As the follower 66 moves up and down as a result of the movement of the rotorcam 64 and tooth 72, the corresponding longitudinal extent of each drive system 40, 42 changes (increases and decreases) causing the corresponding convex surface of the dome 110 to push against and flex the corresponding diaphragm of the corresponding ventricle 32, 34. This, in turn, leads to the respective change of the volume of the corresponding of the right and left ventricles 32, 34: the volume of a ventricle decreases with increase of the longitudinal extend of the drive system 40, 42 and increases with decrease of such longitudinal extent. Such volume variation thereby simulates the contraction and expansion of a human heart and allows blood to flow into, and then be pumped out of, the total artificial heart system 30. The corresponding change in internal pressure of the driving fluid (here, in one example—gas or air) within the drive system 40, 42 may also act as a spring, thereby helping to suction the cam follower 66 back toward the base 50 after the drop-off 88 (shown in and/or discussed in reference to FIG. 7) has passed under the tooth 72.

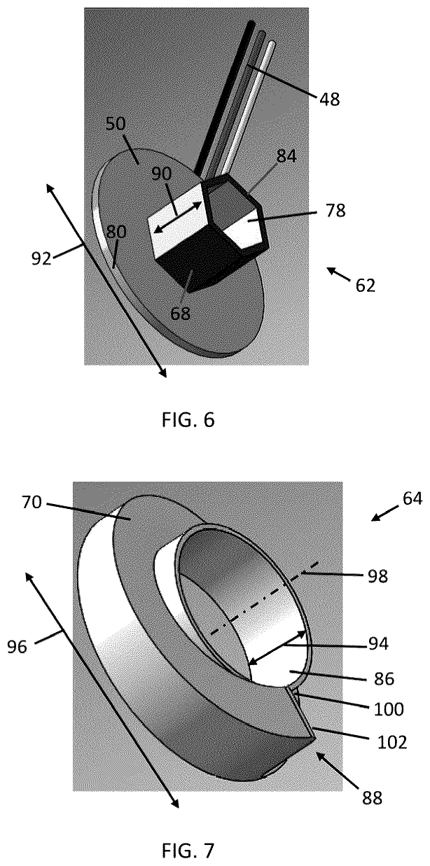

FIG. 6 presents a perspective view of one specific embodiment of the stator assembly 62 including one or more electrical wires 48 coupled to a base portion 50, from which the housing 68 (hexagonal, in this example) extends. The hexagonal housing 68 has a hexagonal cross section, while in related embodiments, the housing 68 may include a square-shaped, rectangular, triangular, elliptical, pentagonal, or otherwise shaped cross-section dimensioned such as to allow the shaft 72 (shown in FIG. 5; which, depending on the situation, is shaped accordingly—hexagonally or otherwise) of the follower 66 to fit within the interior 78 of the housing 68 and slide therewithin, without rotating about the corresponding axis. The interior 78 of the housing 68 is sized and shaped such that its internal geometry substantially matches that of the shaft 74 (while providing for required tolerances between these mating components). FIG. 6 also illustrates a height 90 of the housing 68, as well as a diameter 92 of the base portion 50. In one or more related embodiments, the diameter 92 of the base portion 50 may be from about two to about four times the height 90 of the housing 68. In another embodiment, the diameter 92 of the base portion 50 may be from about 2.5 to about 3.5 times the height 90 of the housing 68; or about three times the height 90 of the housing 68. Depending on the specifics of a particular implementation, the thickness of the wall 84 of the housing 68 (in this specific example—the thickness of the hexagonal wall) may be from about 0.5 millimeters to about 5 millimeters, or from about 0.8 millimeters to about 3 millimeters, or from about 1 millimeter to about 2 millimeters. The stator assembly 62 may also possess an outer lip 80 extending around the outer diameter of the base portion 50. The outer lip 80 is structured to interface with the lower housing 76 (shown in FIG. 5) of the right or left artificial ventricle 32, 34 such as to have the lower (ventricle) housing 76 to extend tightly around the outer lip 80 in a compression fit fashion. The lower housing 76 may also include one or more notches, grooves, slots, slits, and/or other features dimensioned to hold the outer lip 80 to thereby tightly seal the contents of the left and right drive system assemblies 40, 42 between the base portion 50 and the shells of the corresponding ventricles. In operation, neither the lower housing 76 nor the outer lip 80 rotates, remaining stationary. As such, in some embodiments, the lower housing 76 and the outer lip 80 lend themselves to being epoxied, welded, brazed, soldered, glued, adhered, bonded, taped, fused, sintered, and/or otherwise substantially immovably joined to/with one another. Additional features of the stator assembly 62 and motor architecture are illustrated in and discussed in reference to FIGS. 16, 17, and 18 below.

Referring now to discussion of constituent components of the drive systems 40, 42, FIG. 7 depicts, in a perspective view, an embodiment of the substantially monolithic, single-piece or single-component rotorcam 64 including the ramp 70 (which is shown to extend circumferentially about the rotorcam 64 and radially outward of a center bore 86). The center bore 86 includes a hollow, cylindrical space having a height 94 and being concentric about a rotorcam centerline 98. The ramp 70 gradually increases in height, along the circumference of the rotorcam, until it reaches the full height 94 of the rotorcam 64, At this point, the ramp 70 transitions to a drop-off 88, which in turn interfaces with a bottom portion 100 (also illustrated in FIGS. 9, 10, and 11) of the ramp 70. In one specific case, at the transition 102 between the top of the drop-off 88 and the bottom portion 100 of the ramp 70 may have a rounded profile. In various embodiments, a diameter 96 of the rotorcam may be from about two to about ten times the rotorcam height 94; or from about three to about eight times the rotorcam height 94; or from about four to about seven times the rotorcam height 94; or from about five to about six times the rotorcam height 94. In at least one case, the rotorcam 64 is configured to act as a permanent magnet and may include neodymium iron boron (Nd—Fe—B), iron, cobalt, samarium cobalt (Sm—Co), aluminum, alnico, bonded Nd—Fe—B, magnetite, ceramic (hard ferrite), ferrite, gadolinium, rare earth elements, strontium, barium, iron (III) oxide, combinations and alloys thereof, and/or other magnetic materials. By structuring the rotorcam 64 to integrate a rotor for use in an electric motor with a cam, the rotorcam 64 is configured to perform two of these functions (the function or the rotor and the function of the cam) simultaneously while at the same time reducing not only the overall volume of the drive system 40, 42 but also the number of constituent individual parts of the drive system 40, 42.

FIG. 8 depicts the cam follower 66 in a perspective view, illustrating the tooth 72 and the shaft 74 (shown in this example as a hexagonal shaft). The shaft 74, in related embodiments, may include otherwise shaped cross-sections, including but not limited to square-shaped, rectangular, triangular, elliptical, pentagonal, and/or cross-sections shaped otherwise such that the shaft 74 is enabled to fit in and slide within the housing 68 without rotating during the operation of the system. The tooth 72 may include a taper 104 having a generally curved (preferably but optionally—monotonically curved) surface along a leading edge of the tooth as well as a substantially linear (or planar) facet or portion or surface 106 along a trailing edge of the tooth. The linear facet or surface 106—when present—allows the tooth 72 (and the=cam follower 66) to drop down toward the base portion 50 (shown in FIG. 6), after the drop-off 88 (shown in FIGS. 7 and 9, 10, and 11) passes underneath the tooth 72. The presence of the curved surface of the taper 104 along the leading edge of the tooth 72 increases the effective cross-sectional area of the tooth 72, thereby increasing the strength and robustness of the tooth 72, and reducing the likelihood the tooth will become damaged or broken. The tip 108 of the tooth 72 may be additionally or in the alternative rounded or curved (which, when implemented, aids in preventing damage to the tooth 72 and/or other components of the drive system 40, 42).

Referring again to FIG. 8, the cam follower 66 may be structured to include a follower dome 110 (as shown), which, when present, contains a domed curvature and/or gradual contouring (i.e., a larger diameter contouring than the diameter of the cam follower 66). The follower dome 110 may be convex toward (that is, as seen from) the left and/or right artificial ventricle 32, 34 (in which cae such dome is “upwardly convex”), and may form both a barrier and interface between the blood side of the follower dome 110 and the air side (gas side) of the follower dome 110.

At an outer diameter, the carrier dome 110 interfaces with an outer rim 124, which is preferably dimensioned to fit within the cylindrical housing 76. The follower dome 110 is utilized to exchange/transfer pressure between the blood and the air/gas while the follower dome 110 simultaneously acts as a barrier between these two fluids, thereby preventing the blood from ever being exposed to the air/gas, and vice versa. As the follower dome 110 (i.e., and cam follower 66) moves up and down, the volume of the gas/air within each drive system 40, 42 as well as the volume of blood within the right and left ventricles 32, 34 continuously changes. In other embodiments, the cam follower 66 of FIG. 8 may include multiple teeth 72, circumferentially spaced around the cam follower 66 while the rotorcam 64 of FIG. 7 may include multiple ramps circumferentially spaced around the rotorcam 64, and corresponding to the spacing and number of teeth 72 of the cam follower 66. Each of the cam follower 66, the rotorcam 64, and the stator assembly 62 may have about the same outer diameter. In one or more embodiments, each of the cam follower 66 and the rotorcam 64 may include a slightly smaller outer diameter than the stator assembly 62 due to the cam follower 66 needing to translate up and down within the lower housing 76, and due to the rotorcam 64 needing to rotate within the lower housing 76. The base portion 50 may fit tightly within the lower housing 76 as it neither rotates nor translates within the lower housing 76, and thus does not need to move relative to the lower housing 76.

FIG. 9 illustrates a perspective view of the assembled drive system 40, 42 including the stator assembly 62, the rotorcam 64, and the cam follower 66. A clockwise direction 116 (i.e., when viewed from above) indicates the direction in which the rotorcam 64 rotates in operation of the embodiment of FIG. 9. An axial or longitudinal direction 122 (i.e., parallel with the cam centerline 98, shown in FIG. 7) indicates the direction in which the cam follower 66 translates in the embodiment of FIG. 9. The drive system 40, 42 may include a rounded portion 102 defining a transition between the top of the ramp 70 and the drop-off 88, as well as a bottom rounded portion 89 defining a transition between the drop-off 88 and the bottom portion 100. The bottom portion 100 may include a flat portion 112 that transitions into an inclined portion 114. In other embodiments, the bottom portion 100 may include only an inclined portion 114 (i.e., the ramp 70) such that the bottom rounded portion 89 forms a transition between the drop-off 88 and the inclined portion 114 (i.e., without first transitioning to a flat portion 112). The (hexagonal, as shown) shaft 74, in the embodiment of FIG. 9, is partially disposed within the hexagonal housing 68, both being disposed radially within the center bore 86 of the rotorcam 64. Each of the stator assembly 62, the rotorcam 64, and the cam follower 66 may be disposed within the lower housing 76 (i.e., extending down from the left and/or right ventricle 32, 34 (not shown)) or within an alternate cylindrical housing 76. In the embodiment of FIG. 9, the drive system 40, 42 may also include at least one first proximity sensor 118 disposed within the base portion 50 (or outer lip 80) of the stator 62, as well as at least one second proximity sensor 120 disposed within the cam follower 66. The first and second proximity sensors 118, 120 may be used to determine the distance between the base portion 50 and an outer rim 124 of the cam follower 66. This distance is directly proportional to the volume of air (more generally, gas that is being used) within each drive system 40, 42, which in turn is inversely proportional to the volume of blood within the right and/or left ventricle 32, 34, at any given instant. By sensing and tracking the volume of blood being pumped through the total artificial heart system 30 via the first and second proximity probes 118, 120, a total cardiac output or production may be approximated and monitored.

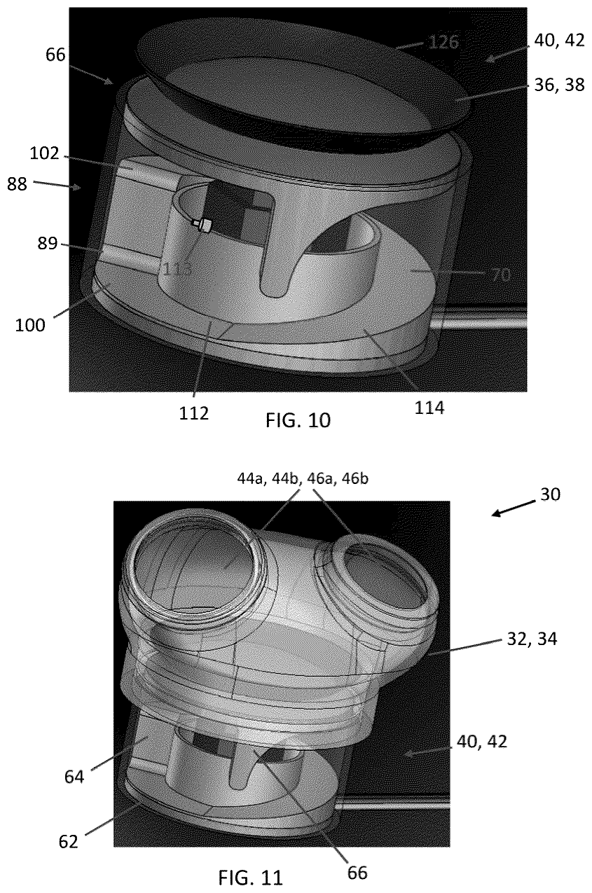

FIG. 10 illustrates an assembled perspective view of the drive system 40, 42 including the stator assembly 62, the rotorcam 64, and the cam follower 66. The cam follower 66 may include the rounded portion 102 at the top of the ramp 70 transitioning to the drop-off 88, which transitions to the bottom rounded portion 89, and then to the bottom portion 100 which comprises the flat portion 112, and the inclined portion 114 (or ramp 70). In the embodiment of FIG. 10, the diaphragm 36, 38 extends from the follower dome 110. The diaphragm 36, 38 may extend both axially upward as well as radially outwardly from the follower dome 110 such that a diaphragm leading edge 126 may make contact with the right or left ventricle 32, 34, thereby providing an extra barrier or seal between the blood chambers (i.e., ventricles 32, 34) and air chambers (i.e., the drive systems 40, 42) of the total artificial heart system 30. The drive system 40, 42 may include one or more pressure sensors 113 disposed on the rotorcam 64 and/or on the stator assembly 62 (for example on the retaining lip 132 of FIG. 17, and/or within the cylindrical housing 128 or stator base portion 50). The one or more pressure sensors 113 may be used to determine the pressure of the air within the drive system 40, 42 as the cam follower 66 rises and falls due to the rotation of the rotorcam 64.

FIG. 11 illustrates an assembled perspective view of the total artificial heart system 30 with the right and/or left ventricle 32, 34 coupled to the drive system assembly 40, 42 including the stator assembly 62, the rotor cam 64, and the cam follower 66. FIG. 11 also illustrates the locations of one or more artificial valves including the tricuspid 44a, the bicuspid 46a, the pulmonary 44b, and the aortic 46b. In each of the embodiments of FIGS. 9-11, the one or more teeth 72 of the cam follower 66 would generally be in contact with the one or more ramps 70 of the rotorcam 64 while the drive system 40, 42 is in operation.

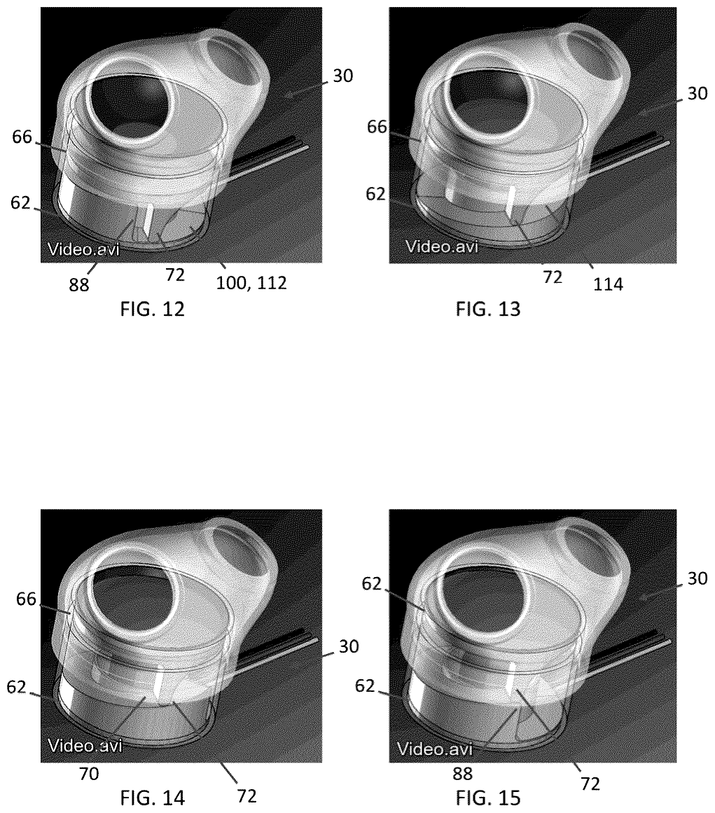

FIGS. 12, 13, 14, and 15 show progressive perspective views of the total artificial heart system 30 in operation, according to aspects of the present embodiments. In the embodiment of FIG. 12, the at least one tooth 72 is at the flat portion 112 of the bottom portion 100 at the bottom of the drop-off 88. In the embodiment of FIG. 13, the at least one tooth 72 is on the inclined portion 114 (or ramp 70). In the embodiment of FIG. 14, the at least one tooth 72 is close to the top of the ramp 70. In the embodiment of FIG. 15, the at least one tooth 72 is at the very top of the ramp 70, at the edge of the drop-off 88. In the embodiments of FIGS. 12-15, the cam follower 66 gets progressively further away from the stator assembly 62, which corresponds to blood being pushed by the cam follower 66 (and diaphragm 36, 38) out of the right or left ventricle 32, 34. The cam follower 66 does not rotate with the rotorcam because the hexagonal housing 68 does not permit the hexagonal shaft 74 to rotate therewithin. The housing 68 and shaft 74 may similarly be square-shaped, triangular, and other shapes (i.e., other than circular) such that the housing 68 will hold the shaft 74 but prevent it from rotating with the rotorcam 64.

In operation, the right and left ventricles 32, 34 are only partially filled during normal conditions, in order to provide extra capacity when the blood flow in the body of the patient is increased as a result of physical activity. In one embodiment, the air side (or, more generally, gas side) of each ventricle has a volume of approximately from about three (3) to about six (6) cubic inches, or from about four (4) to about five (5) cubic inches, or from about 4.1 to about 4.7 cubic inches). Each of the diaphragms 36, 38 may have an area from about four (4) to about eight (8) square inches, or from about five (5) to about seven (7) square inches, or from about 5.7 to about 6.6 square inches. The compression ratios of each drive system 40, 42 on the air side (or gas side, more generally; i.e., ratio of maximum volume to minimum volume for a given operating condition) varies from one operating mode to the next. For example, in a maximum blood volume case (i.e., strenuous exercise), the compression ratio may be about three (3) to one (1), or from about 2.5 to about 3.5 to one (1). In a moderate exercise or activity case, the compression ratio may be about to (2) to one (1), or from about 1.75 to about to 2.5 to one (1). In a resting or sleeping mode of operation, the compression ratio may be about 1.5 to one (1) or from about 1.25 to about 1.75 to one (1).

Each of the components of the drive system 40, 42 including the stator assembly 62, the rotorcam 64, the cam follower 66, the wires 48, as well as the right and left artificial ventricles 32, 34 may be coated with an inert material that does not cause a reaction or response within the human body. In one embodiment, each of the components of the drive system 40, 42 may be coated with a segmented polyurethane solution (SPUS) that is both biocompatible (or bio-inert) within the human body (i.e., completely inert), and that is also both fatigue and strain resistant. In one or more embodiments, right and left artificial ventricles 32, 34 may be composed of SPUS. The SPUS components may be formed via one or more molding processes as separate components and then subsequently glued together. In other embodiments, various components or subcomponents may be formed of SPUS via a single injection-molding process. One or more components and/or sub-components of the present disclosed embodiments may also be composed of or coated with other synthetic materials, such as silicone rubber, thermoplastic elastomers (TPE), and/or polyvinyl chloride (PVC). The SPUS material used in connection with the present disclosed embodiments may be able to accommodate tensile loads from about 5000 psi to about 8000 psi, and may be able to accommodate stiffness (i.e., modulus) loadings from about 400 psi to about 800 psi or from about 450 psi to about 750 psi. By using SPUS materials (either as the underlying component materials or as coatings on components that are composed of other materials) in connection with the components of the present disclosed embodiments, reactions and other responses occurring from exposure of one or more biological tissues to the components described herein may be avoided.

### BLDC Motor Architecture

FIGS. 16, 17, and 18 illustrate embodiments of the stator assembly 62 (FIGS. 16 and 17) and an electric motor assembly 140 (FIG. 18), according to aspects of the present embodiments. Referring to FIG. 16, the stator assembly 62 may include a plurality of winding groups 130 each oriented longitudinally and spaced around the hexagonal housing 68. Each of the winding groups 130 may include longitudinally aligned conductive coil wires 138 wound around one or more cores (not shown; i.e., solid frames, supports, and/or features that provide a structure around which the wires 138 may be wound). In the embodiment of FIG. 16, six (6) stator winding groups 130 are included, but electric motors according to the present embodiments (including brushless direct current (BLDC) motors) may include other numbers of windings including, for example, eight, (8), nine (9), twelve (12), twenty-four (24), forty-eight (48), as well as other numbers of stator windings 130. Each of the stator winding groups 130 may be composed of silicon core iron, copper, aluminum, and other materials. Opposing winding groups 130 (for example, the first and fourth, the second and fifth, and the third and sixth winding groups 30 in the embodiment of FIG. 16) may be electrically coupled such that they provide opposing polarity (i.e., north or south) to the surrounding rotorcam 64 (not shown). Adjacent sets of opposing windings 130 may then be electrically activated in succession such that each set imparts an electromagnetic force on the rotorcam 64, causing it to rotate about the stator assembly 62.

Referring again to FIG. 16, the stator assembly 62, may also include a thicker base 50 to accommodate an integral battery system, motor controls and/or circuitry, as well as other components. The stator assembly 62 may also include one or more Hall effect devices or Hall sensors 136 disposed within the base 50 or radially within the cylindrical housing 128. The one or more Hall sensors 136 may be used to sense the presence of a strong and/or changing electromagnetic field (i.e., from the rotorcam 64 as it rotates past), in order to determine the rotational position of the rotorcam 64 (and thus the rotational speed as well). The Hall sensors 136 may also be used to control the rotational speed of the rotorcam 64 (i.e., overall revolutions per minute or radians per second, for example), as well as the instantaneous angular speed within each revolution, which also may be selectively controlled.

FIG. 17 illustrates a perspective view of the stator assembly 62 according to aspects of the present embodiments. The stator assembly 62 may include one or more springs 134 disposed within the hexagonal housing 68. The spring may be coupled to both the stator base 50, as well as the cam follower 66 (shown in FIGS. 5 and 8-15) for pulling the cam follower 66 back toward the stator base 50 when the one or more teeth 72 (shown in FIGS. 5 and 8-15) passes the dropoff 88 (shown in FIGS. 5 and 8-15). Stated otherwise, one end of each spring 134 is coupled to the bottom of the hexagonal housing 68 while the other end of each spring 134 is coupled to the top of the hexagonal shaft 74. The one or more springs 134 are disposed within both the hexagonal housing 68 and the hexagonal shaft 74. The spring 134 expands as the rotorcam 64 pushes the tooth 72 and cam follower 66 upward. The one or more springs 134 then pull the cam follower 66 back toward the stator assembly 62 after the drop-off 88 passes under the tooth 72.

Referring again to FIG. 17, the stator assembly 62 may also include a retaining lip 132 extending radially outwardly from the upper circumference of the cylindrical housing 128. The retaining lip 132 may be ring-shaped and may be concentric about the cylindrical housing 128. The retaining lip 132 may be used to hold the rotorcam 64 to the stator assembly 62, thereby allowing the rotorcam 64 to rotate about the cylindrical housing 128, while preventing the rotorcam 64 from translating upwardly and/or downwardly (i.e., along the longitudinal direction). In embodiments that do not include a retaining lip 132, the rotorcam 64 may remain biased against the stator base 50 via the one or more teeth 72 (shown in FIGS. 5 and 8-15), which is pulled downwardly via the one or more springs 134. Because the hexagonal housing 68 is radially disposed within the one or more winding groups 130, the hexagonal housing 68 may also be termed an inner stator housing 68. Similarly, because the cylindrical housing 128 is disposed radially outward of the one or more winding groups 130, the cylindrical housing 128 may also be termed an outer stator housing 128.

FIG. 18 illustrates a top view of a motor assembly 140 (for example, a BLDC motor assembly), according to aspects of the present embodiments. As illustrated in FIG. 18, the motor assembly 140 may include the rotorcam 64 disposed about the cylindrical housing 128, and longitudinally held in place via the retaining lip 132. The rotorcam 64 may include the ramp 70, which may include the rounded portions 102, 89 at the drop-off 88, as well as the flat portion 112, and the inclined portion 114. Disposed radially within the cylindrical housing 128, the motor assembly 140 may include multiple winding groups 130, the hexagonal housing 68, the hexagonal shaft 74, and the one or more springs 134. One or more wires 48 may also be electrically coupled to the motor assembly 140. In the embodiment of FIG. 18, the motor assembly 140 may include one or more dampers 142 disposed within the hexagonal housing 68 at the bottom of the hexagonal housing 68. The one or more dampers 142 may be compressible, and may extend up to only a fraction or partial length of the height 90 of the hexagonal housing 68. For example, the damper 142 may extend up to only about five (5) percent to about fifty (50) percent of the height 90 of the hexagonal housing 68. In other embodiments, the damper 142 may extend to from about ten (10) percent to about forty (40) percent of the height 90 of the hexagonal housing 68. In other embodiments, the damper 142 may extend to from about fifteen (15) percent to about thirty (30) percent of the height 90 of the hexagonal housing 68. In other embodiments, the damper 142 may extend to from about twenty (20) percent to about twenty-five (25) percent of the height 90 of the hexagonal housing 68.

Referring still to FIG. 18, in operation, the one or more dampers 142 prevents the cam follower 66 (and one or more teeth 72 thereof) from slamming too violently or rapidly back down onto the bottom portion 100 (for example onto the flat portion 112) of the rotorcam 64 (i.e., after the drop-off 88 passes under the one or more teeth 72). As such, the one or more dampers 142 may prevent wear and tear of the drive system 40, 42, and may also allow for a more gradual operation of the cam follower 66 within the right and/or left ventricle 32, 34. The central venous pressure helps fill the heart (with blood) each time the tooth 72 drops off the drop-off 88. The damper 142 helps prevent a high change in pressure too quickly and ensures a more gentle and gradual ramp drop-off. For example, by using the damper 142, pressure drops (i.e., dP/dT) above a certain threshold may be avoided. The motor 140 may also be used to prevent rapid changes in pressure. Because the motor 140 may be selectively controlled to any rotational speed (even within a single rotation), multiple rotational speeds may be chosen during each of the systole and diastole phases of each cycle. For example, during the systolic phase when the right and left ventricles 32, 34 are being evacuated, the motor 140 may be controlled such that the rotational speed is higher while the tooth or teeth 72 are on the inclined portion 114 of the ramp (thereby raising the cam follower 66 and contracting the ventricles 32, 34 quickly). The motor 140 may then be controlled to a slower speed for the beginning of the diastolic phase (when the ventricles 32, 34 are expanding and filling), allowing the tooth 72 and cam follower 66 to drop all the way back down to the flat portion 112. In one or more embodiments, the motor 140 may be controlled such that transitions between faster and slower rotational speeds are made gradually and/or gently so that the artificial valves 44a, 44b, 46a, 46b do not experience excessive stresses by opening or closing too quickly. By gradually transitioning between slower rotational speeds and faster rotational speeds (and vice versa), “water hammer” effects may be avoiding, thereby reducing potential damage to the artificial valves 44a, 44b, 46a, 46b themselves (i.e., by gently opening and closing them), but also reducing damage to the surrounding blood. In addition, fluid shear stress (i.e., within the blood) may be minimized by limiting the pressure drop (i.e., dP/dT) by controlling the rotational speed of the rotorcam 64.

In operation, anywhere from one (1) to five (5) or six (6) inch-pounds of torque may be required to rotate the drive system 40, 42, (the higher end of the torque range corresponding to a hypertensive patient whose blood pressure will externally act on the drive system 40, 42, thereby causing increased resistance and friction within the system). As a result, the power of the electric motor 140 required to power the system may be from about one (1) watt to about ten (10) watts or from about two (2) watts to about seven (7) watts, or from about three (3) watts to about six (6) watts, or from about four (4) watts to about five (5) watts. In other embodiments, the electric motor 140 may also run within other suitable power ranges.

### Related Drive System Embodiments

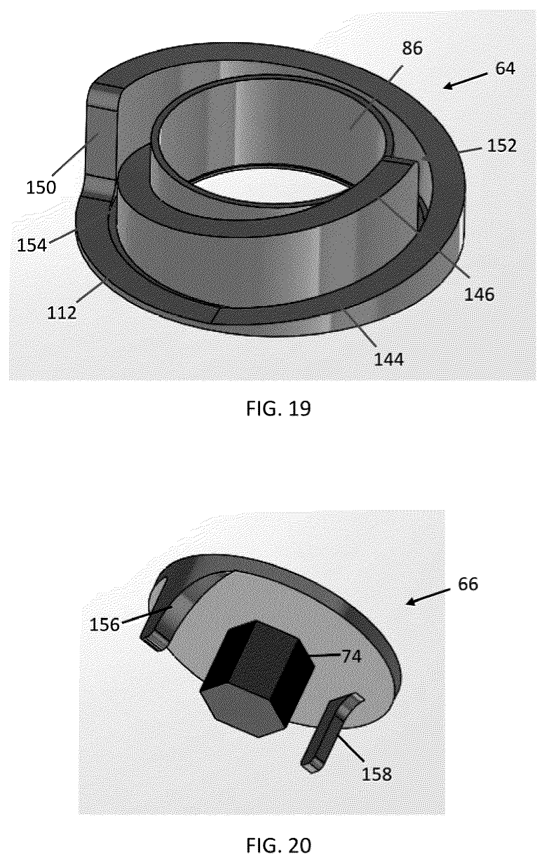

FIG. 19 illustrates a perspective view of a specific embodiment of the single-piece, monolithic rotorcam 64 in a dual ramp configuration, according to the idea of the present invention. As shown, the monolithic rotorcam 64 includes an outer ramp 144 and an inner ramp 146 that is substantially concentrically disposed within the outer ramp 144 (and disposed radially outwardly with respect to the rotorcam center bore 86). The outer and inner ramps 144, 146 (each of which is configured as cams of the cam-and-follower system (64+66) include outer and inner drop-offs 150, 152 angularly clocked by a predetermined angle (in one preferred embodiment—about 180-degrees) apart from each other, as viewed along the axis of the rotorcam. (The skilled person will readily appreciate that, when the predetermined angle is chosen to be about 180 degrees or, better yet, precisely 180 degrees, the operation of the cam-and-follower portion of the drive system 40, 42 necessarily ensures rhythmical 50-percent duty cycle systole and diastole of the operational cycle of the ventricle 32, 34 when the system 40, 42 is operated in conjunction with the ventricle 32, 34—whether implanted in the chest of the recipient patient or not.) The outer and inner ramps 144, 146 may also include outer and inner flat portions 112, also disposed 180-degrees apart from each other. In the embodiment of FIG. 19, each of the flat portions 112 span approximately 90-degrees of the circumference of the rotorcam 64, while the inclined portions of each of the outer and inner ramps 144, 146 span approximately 270-degrees of the circumference of the rotorcam 64. In other embodiments, each of the flat portions 112 may span from about 0-degrees to about 60-degrees of the circumference of the rotorcam 64, while the inclined portions of each of the outer and inner ramps 144, 146 may span from about 300-degrees to about 360-degrees of the circumference of the rotorcam 64. In other embodiments, each of the flat portions 112 may span from about 60-degrees to about 90-degrees of the circumference of the rotorcam 64, while the inclined portions of each of the outer and inner ramps 144, 146 may span from about 270-degrees to about 300-degrees of the circumference of the rotorcam 64.

Referring again to FIG. 19, the rotorcam 64 may also include a thin radial gap 154 disposed radially outward of the inner ramp 146 and radially inward of the outer ramp 144. Each of the outer and inner ramps 144, 146 may be sloped such that the increase in height as a function of the circumferential angle (or rotation) of the rotorcam 64 is constant. Stated otherwise, each of the outer and inner ramps 144, 146 transitions from a minimum height (i.e., at the flat portion 112) to a maximum height (i.e., just before each drop-off 150, 152) through a rotation of about 270-degrees. As such, the increase in height as a function of angle is constant for both the outer and inner ramps 144, 146. As a result, the increase in height as a function of distance traveled on each ramp is higher on the inner ramp 146 than on the outer ramp 144 since the inner ramp 146 increases the same overall height over a shorter distance.

FIG. 20 illustrates a perspective view of a cam follower 66 in a dual tooth configuration, according to aspects of the present embodiments. The dual-tooth cam follower 66 of FIG. 20 may include an outer tooth 156 as well as an inner tooth 158 disposed at a smaller radius than the outer tooth 156, and circumferentially spaced approximately 180-degrees from the outer tooth 156. Each of the outer and inner teeth 156, 158 are disposed radially outward of the hexagonal shaft 74. In addition, each of the outer and inner teeth 156, 158 are disposed at radii that correspond with those of the outer and inner ramps 144, 146 of the rotorcam 64 of FIG. 19. As such, the outer tooth 156 of FIG. 20 is disposed at the same radius as the outer ramp 144 of FIG. 19, while the inner tooth 158 is disposed at the same radius as the inner ramp 146. In operation, because both the outer and inner teeth 156, 158 interface with at least one of the outer and inner ramps 144, 146, the longitudinal forces acting on the cam and follower system may be more evenly distributed in a dual-tooth/dual-ramp configuration than in a single-tooth/single ramp configuration. Tri-tooth/tri-ramp, quad-tooth/quad-ramp, and other configurations are also possible, in accordance with the present embodiments.

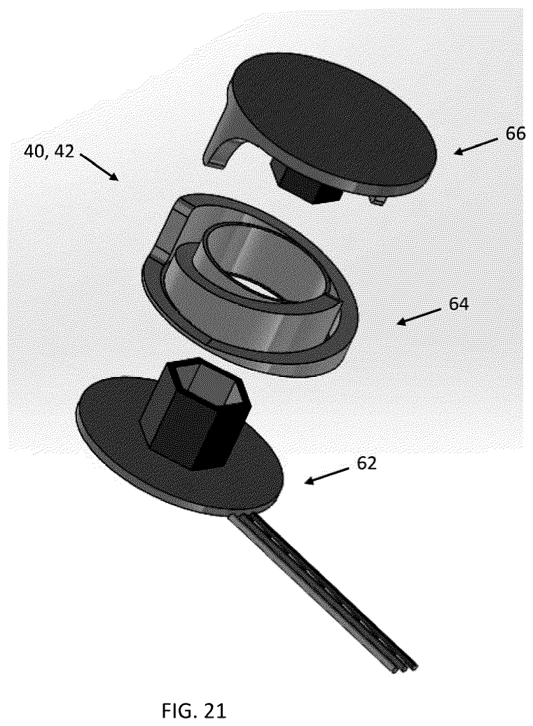

FIG. 21 illustrates a perspective view of a disassembled drive system 40, 42 in a dual ramp/dual tooth configuration including the stator assembly 62, the rotorcam 64, and the cam follower 66, according to aspects of the present embodiments.

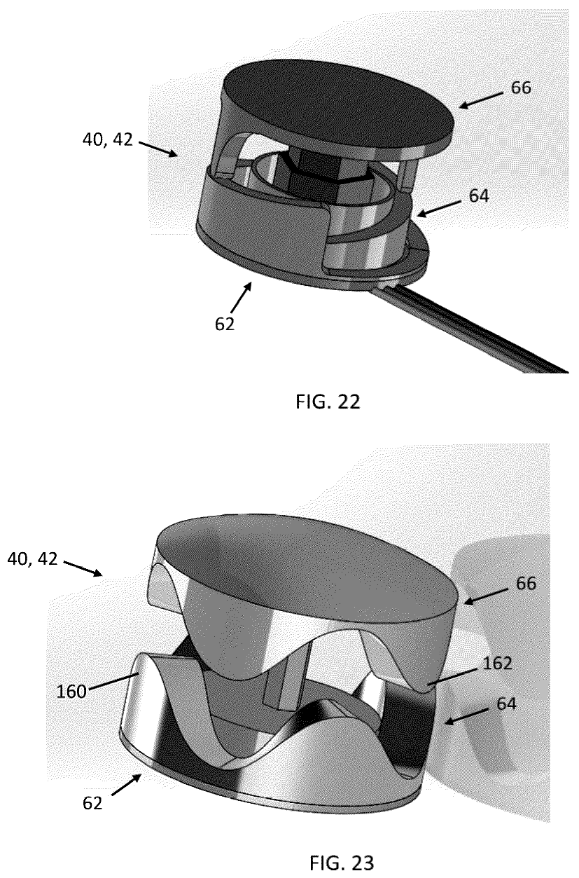

FIG. 22 illustrates a perspective view of an assembled drive system 40, 42 in a dual ramp/dual tooth configuration including the stator assembly 62, the rotorcam 64, and the cam follower 66, according to aspects of the present embodiments.

FIG. 23 illustrates a perspective view of a related embodiment of the drive system 40, 42 in an “emperor crown” configuration that includes the stator assembly 62, the rotorcam 64, and the cam follower 66, in which the rotorcam 64 and the cam follower 66 are shown to be axially separated by a non-zero distance, to illustrate edge surfaces of the angled ramps of the rotorcam and the cam follower that are dimensioned to be substantially congruent with one another. The first edge surface of the rotorcam (the one facing the circular base on the stator assembly 62) is substantially congruent with the surface of the circular base on which this first edge surface rests. In the embodiment of FIG. 23, the rotorcam 64 is structured to be monolithic and to possess a corresponding angled cam ramp circumferentially extending around the axis of the rotorcam and containing one or more cam lobes 160, each cam lobe 160 being contoured such that the rotorcam 64 raises the cam follower 66 as the rotorcam 64 rotates. The cam follower 66 also contains a corresponding angled cam follower ramp circumferentially extending about the axis of the cam follower and including one or more follower lobes 162. Each present cam lobe 160 and each present cam follower lobe 162 is preferably shaped such as to have the edge surface of the angled cam follower ramp and the edge surface of the angled cam ramp (which is the second edge surface of then monolithic rotorcam facing away from the circular based of the stator assembly 62) be substantially congruent with one another. (In other words, due to matching contouring of the cam lobes 160 and the cam follower lobes 162 the cam-and-follower system of the drive system 40, 41, when assembled has these two edge surfaces separated by a substantially non-zero distance in a rest orientation. In the embodiment of FIG. 23, the rotorcam 64 is shown to include four cam lobes 160 while the cam follower 66 is shown to include four follower lobes 162. In other embodiments, each of the rotorcam 64 and cam follower 66 may include one or multiple numbers of cam lobes 160 and/or follower lobes 162. In one or more embodiments, each of the cam follower lobes 162 may include one or more rollers (not shown) to help facilitate movement of the rotorcam lobes 160 there under. A low friction, wear-resistant coating may also be disposed on each of the cam and follower lobes 160, 162 to minimize contact friction between the rotorcam 64 and the cam follower 66. Notably, in at least one implementation of the drive system 40, 42 that includes a cam-and-follower system shown in FIG. 23, the spatial profiles of the angled cam ramp and of the cam follower ramp are dimensioned to have multiple maxima and multiple minima, with all of the maxima being separated from the circular base on the stator assembly by the same maximum axial distance and with all of the minima being separated from such circular base by the same minimum but nonzero axial distance (as shown in the example of FIG. 23. Generally, however, when multiple cam lobes are present in the angled cam ramp, such lobes have different heights. In at least one specific case, each of the angled cam ramp and the angled camp follower ramp is closed upon itself around a corresponding circumference (of the rotorcam and cam follower, respectively).

### Power Supply and Charging System

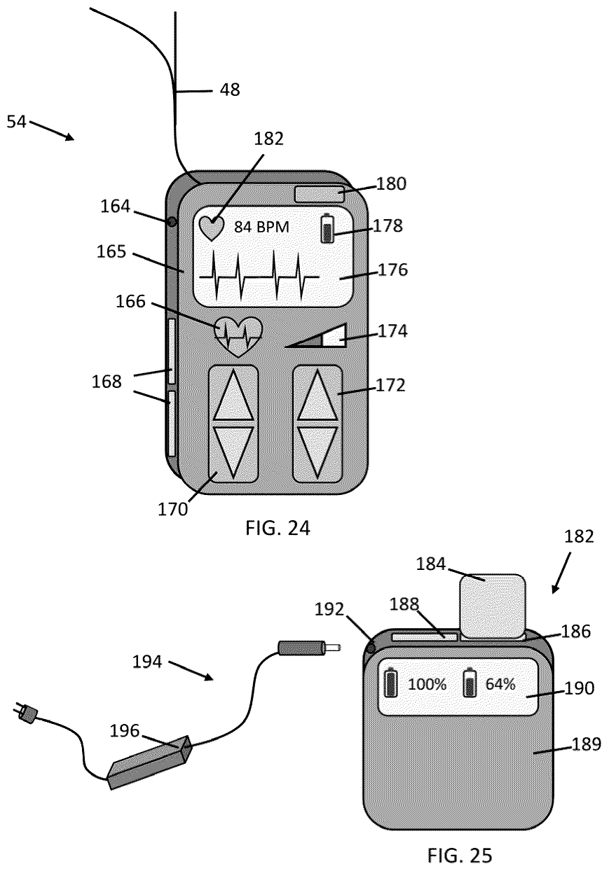

FIG. 24 illustrates a perspective view of a mobile power supply 54 according to aspects of the present disclosed embodiments. The mobile power supply 54 may be electrically coupled to one or more wires 48 (or groups of wires) for supplying power to the electric motor assemblies 140 of each of the drive systems 40, 42. The mobile power supply 54 may also include a mobile power supply housing 165 and at least one external charging port 164 (for example, for plugging the mobile power supply 54 directly into a wall power outlet) disposed within the housing 165. The mobile power supply 54 may also include one or more battery ports 168 disposed in the housing 165 for inserting rechargeable batteries into the mobile power supply 54. In the embodiment of FIG. 24, the mobile power supply 54 includes two (2) battery ports 168, which allows at least one battery to be installed in the mobile power supply 54 to provide power thereto at all times, even while a second battery is being charged. The external charging port 164 may be used, in one or more embodiments and/or use cases, to simultaneously power the drive systems 40, 42, while also charging the batteries. The mobile power supply 54 may also include a heartrate control module 166 including a first set of control buttons 170 for adjusting (i.e., increasing or decreasing) the heartrate. In accordance with the present embodiments, each heartbeat may be defined as one complete cycle of the cam follower 66 rising up a rotorcam 64 ramp 70, then falling off the drop-off 88 (which corresponds to the contraction and expansion of the right and/or left ventricle 32, 34). As such, in a single tooth/single ramp embodiment (i.e., similar to FIG. 9), the drive system 40, 42 will create one heartbeat for each 360-degree rotation of the rotorcam 64. Similarly, in a dual tooth/dual ramp embodiment (i.e., similar to FIGS. 21 and 22) the drive system 40, 42 will create one heartbeat for each 360-degree rotation of the rotorcam 64. In other dual tooth/dual ramp embodiments in which each ramp 70 spans one-hundred and eighty (180) degrees and both teeth 72 interface with (and are disposed over) both ramps 70, the drive system 40, 42 will create two heartbeats for each 360-degree rotation of the rotorcam 64. The heartrate, then, as it applies to the total artificial heart system 60 of the present embodiments, is simply the number of heartbeats (as defined above) per minute.

Referring still to FIG. 24, in order to increase the cardiac output of the system, the patient may use the first set of control buttons 170 to increase (or alternatively decrease) the heartrate of the total artificial heart 30. The mobile power supply 54 may also include a volume control module 174 including a second set of control buttons 172 for adjusting (i.e., increasing or decreasing) the volume (that is, the volume of pumped blood) of each heartbeat. For example, by allowing the cam follower 66 to travel all the way to the bottom of the drive system 40, 42 (i.e., near the base 50), each of the right and left ventricles 32, 34 may expand to a larger volume,

thereby allowing more blood to flow into each of the right and left ventricles 32, 34, to be pumped to the pulmonary or circulatory systems. As such, both the heartrate control module 166 and the volume control module 174 may be used to increase or decrease the overall cardiac output (or production). The mobile power supply 54 may also include a display screen 176 for displaying various data and information such as one or more battery charge levels 178, a heartrate indication 182, as well as other information. The mobile power supply 54 may also include a wireless communications module 180 allowing the mobile power supply 54 to wirelessly connect to Wi-Fi-enabled devices and/or network-connected devices such as smart phones, laptops, tablets, desktop computers, networks, servers, chargers, and/or other devices. The mobile power supply 54 may also include one or more clips, bands, or other connection features (not shown) enabling wearing (for example by clipping to a belt) and carrying of the mobile power supply 54. By including various features in the mobile power supply 54, the mobile power supply 54 includes an integral control interface operatively coupled to the motor assembly 140 and/or the stator assembly 62.

FIG. 25 illustrates a perspective view of a charging system 182, in accordance with the present disclosed embodiments. The charging system 182 may at least one housing 189 including first battery port 186 and a second battery port 188 for receiving one or more rechargeable batteries 184, and for electrically coupling them with an external power supply 194. The external power supply 194 may include one or more alternating current (AC) to direct current (DC) converters 196 such that the external power supply 194 can receive AC power (for example, from a wall outlet) and also supply DC power to the total artificial heart 30 via the one or more batteries 184. The charging system 182 may include one or more charging ports 192 disposed within the housing 189 for plugging in the external power supply 194. The charging system 182 may also include a display screen 190 disposed within the housing 189 for displaying data and other information such as the charge levels of the one or more batteries 184.

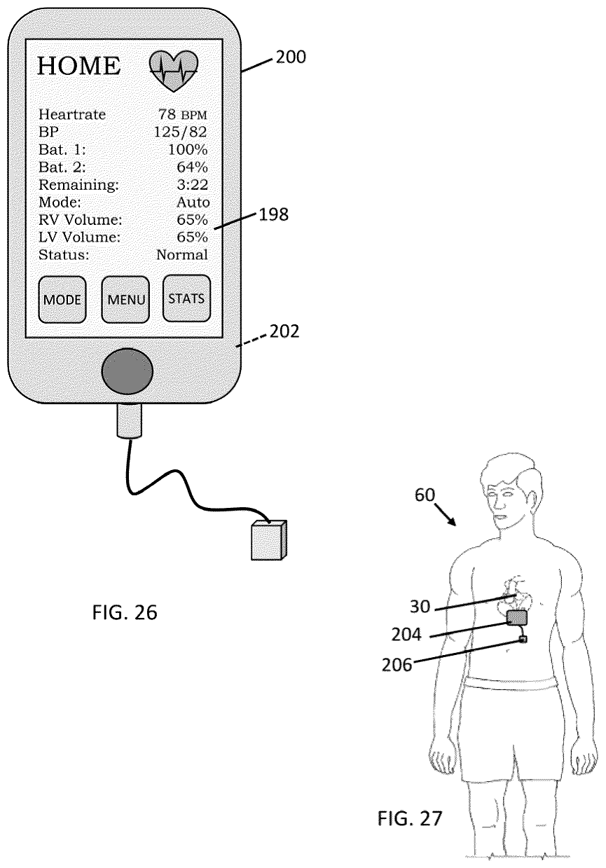

FIG. 26 illustrates a front view of application software 198 that may be displayed on one or more electronic devices such as a smart phone, desktop computer, laptop computer, tablet, and/or other device 200. The application software 198 may display such information as the heartrate, the blood pressure, a first and second battery charge level, the remaining time until both batteries are depleted (i.e., remaining life of the one or more batteries), a control mode, a right and left ventricle pumping volume, as well as an overall system status (for example, such as normal, alert, caution, etc.). A communication module 202 of the electronic device 200 may be communicatively coupled to the communication module 180 of the mobile power supply 54 such that data and information from the mobile power supply 54 may be transmitted to the electronic device 200 (for example wirelessly) in real-time or near-real time (for example in 1, 5, 10, 20, 30, or 60 second increments, etc.). The application software 198 may also include one or more buttons including (but not limited to) a mode button, a stats button (for example, to retrieve statistical operational data), as well as a menu button. The mode button may allow a user to select a control mode such as: 1) maintaining a constant heartrate and varying only the pumped volume of blood within the right and left ventricles 32, 34 when a change in cardiac output is desired; 2) maintaining a constant volume and changing the heartrate when a change in cardiac output is desired; or 3) changing both the volume and the heartrate in a “hybrid” mode of operation.

Referring still to FIG. 26, the application software 198, via the communication coupling with the mobile power supply 54, may (in one or more embodiments) allow the user to change the operation of the total artificial heart 30 directly from the electronic device 200. For example, the application software may enable a user to choose other modes of operation indicative of an overall patient level of activity such as “resting,” “moderate,” and/or “active.” The electronic device 200 may then send a signal to the mobile power supply 54 (which in many embodiments also acts as a control unit and/or a control interface) to increase the cardiac output (for example by increasing the heartrate) when a user has selected an “active” mode of operation. The application software 198 may also include a mode of operation that allows the cardiac output to automatically follow a patient breathing rate, which may be determined via one or more accelerometer or motion sensors 82 (shown in FIG. 4) disposed within the mobile power supply 54 that sense the expansion and contraction of the lungs, stomach, and/or body cavity or diaphragm via direct contact (for example, when the mobile power supply 54 is attached to a belt of the patient). Stated otherwise, by using one or more accelerometer or motion sensors 82 mounted near a patient's waist to determine how quickly or vigorously a patient is breathing, an overall activity level of the patient may be determined and/or approximated, and a cardiac output may accordingly be adjusted to match the patient breathing rate. The application software 198, in one or more embodiments, may also be run on multiple devices simultaneously such that a friend or family member of the patient and/or a remote monitoring team can monitor the patient's status, and take appropriate action if an emergency or potentially problematic operating condition arises.

FIG. 27 illustrates a front view of a total artificial heart system 60 according to aspects of the present embodiments. In the embodiment of FIG. 27, the total artificial heart system 60 may include an implanted total artificial heart 30 including one or more drive systems (in accordance with the present embodiments) electrically coupled to an implanted power supply 204. The implanted power supply 204 may include one or more rechargeable batteries implanted within the patient, and housed, for example, in the thicker base 50 illustrated in FIG. 16. The implanted power supply 204 may be connected to one or more external charging ports 206, which is external to the body. The external charging port allows the implanted power supply 204 to be recharged by connecting an external power supply or charger (such as those illustrated in FIGS. 4, 24, and/or 25) to the external charging port 206. By implanting a power supply 204 within the patient, the patient is afforded periods of time when he or she may be completely disconnected from an external power supply, thereby enabling periods of even greater flexibility for the patient.

### Operational Characteristics

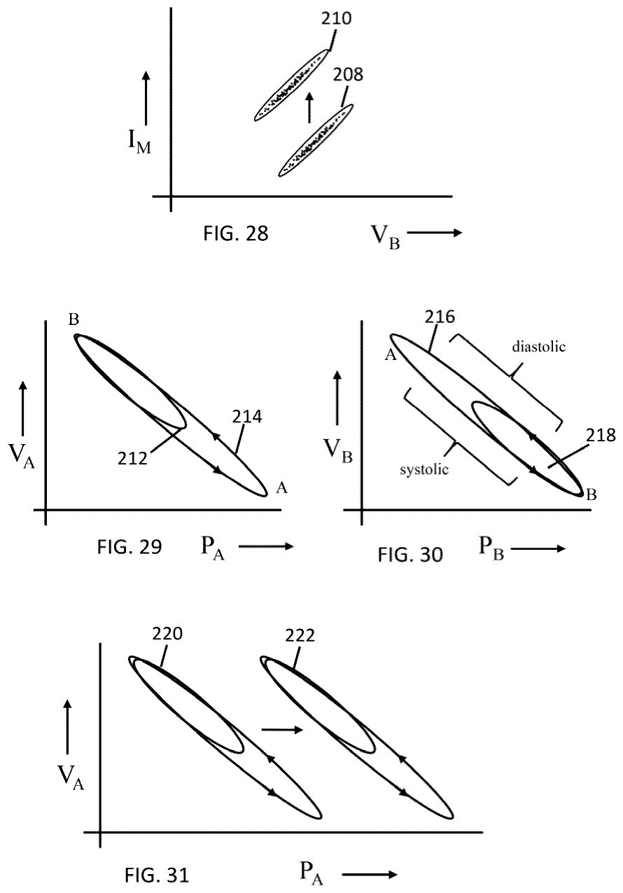

FIG. 28 illustrates a schematic of an electrical current versus volume of blood characteristic, according to aspects of the present embodiments. As the volume of blood VB being pumped increases, the electrical current drawn by the motor 140 IM also increases. The relationship between VB and IM is approximately linear, but may vary based also on the heartrate, as well as based on small variation in the blood temperature (which generally will be maintained within a narrow temperature range by the human body). As the temperature of the blood changes, even slightly, small changes in the viscosity of the blood (and thus the power required to pump the blood) may also occur. The chemistry of the blood, which may also vary over time, may also have minor effects on the viscosity of the blood. Thus, data is expected to accumulate in scattered clusters (which may be approximately linear), similar to a first cluster 208 illustrated in FIG. 28 (i.e., rather than in a strictly linear fashion). If the cluster shifts over time such that data now accumulates in a second cluster 210 where more electrical current IM is required to pump the same volume of blood VB, it may be an indication that either the drive system 40, 42 (or other component of the total artificial heart 30) has degraded and is now operating less efficiently, or possibly that the blood pressure of the patient has increased, thereby causing increased resistance to flow, and requiring additional motor power to pump the same volume of blood. As such, alerts can be triggered and appropriate action can be taken if the IM versus VB cluster has shifted over time. The motor current IM may be measured at the motor assembly 140, or at the mobile power supply 54, while the volume of blood VB being pumped each heartbeat may be derived from the minimum and maximum volumes of air (or, more generally, gas) in the drive system 40, 42, which may be calculated directly from the distance between proximity sensors 118, 120 (shown in FIG. 9). The volume of blood VB being pumped each heartbeat may also be approximated from the input control setting (i.e., the volume of blood that is pumped each heartbeat, as selected at the volume control module 174 of the mobile power supply 54 (FIG. 24)).

FIG. 29 illustrates a schematic of volume of air versus pressure of air characteristic, according to aspects of the present embodiments. The volume of the air VA within the drive system 40, 42 may be determined using the one or more proximity sensors 118, 120, while the pressure of the air PA may be determined via the at least one pressure sensor 113 (shown in FIG. 10). Because the drive system 40, 42 is a closed, air-tight system, the pressure of air may also be derived from the volume of the air within the drive system 40, 42 VA using Boyle's law (or ideal gas law), PV=nRT, where P is the pressure, V is the volume, n is the number of air molecules (which is constant and doesn't change), R is a constant, and T is approximately constant since the human body temperature is maintained within a narrow temperature range. Therefore, pressure and volume (as it applies to the air in the drive system 40, 42) are more or less inversely proportional to one another, and one may be approximated directly from the other. As the volume goes up, the pressure goes down. As the volume goes down, the pressure goes up. Curve 212 (shown in FIG. 29) illustrates the volume vs pressure characteristic for air in the drive system 40, 42 when the system is set to pump a smaller volume of blood. In the operating condition corresponding the curve 212, because the cam follower 66 does not drop all the way to the bottom portion 100, the volume of air (i.e., under the cam follower 66) does not get squeezed down as much. As a result, the drive system 40, 42 goes from the maximum volume of air down to a medium volume of air, back to a maximum volume of air (and the air pressure correspondingly goes from a minimum pressure to a medium pressure, back to a minimum pressure). In the operating condition corresponding to curve 214, the drive system 40, 42 is set to allow the cam follower 66 to drop all the way down to the bottom portion 100, thereby squeezing the air down to a minimum volume and allowing each of the right and left ventricles 32, 34 to expand to a maximum volume (corresponding to a maximum cardiac output). In the operating condition corresponding to curve 214, the drive system 40, 42 goes from the maximum volume of air down to a minimum volume of air, back to a maximum volume of air (and the air pressure correspondingly goes from a minimum pressure to a maximum pressure, back to a minimum pressure).

Referring still to FIG. 29, the pressure and volume may be inversely proportional in a strictly linear relationship under steady-state conditions (that is, assuming a constant temperature). However, under typical operating conditions of the drive system 40, 42, the volume is continuously changing and thus the conditions are never truly steady-state. As such, the pressure may lag the volume slightly, at least as sensed by the one or more pressure sensors 113. Stated otherwise, as the volume changes, it takes time for the pressure to “catch-up” such that the pressure sensor 113 “reacts” and sends a signal reflective of the new pressure. This pressure sensor 113 time lag is reflected by the elliptical shape of the curves 212, 214, 216, 218, 220, and 222 in FIGS. 29-31. In other embodiments, the volume vs. pressure relationship may be represented as strictly linear and inversely proportional. Arrows on one or more of the curves indicate how the volume versus pressure relationship changes when the system is in operation.

FIG. 30 illustrates a schematic of a volume of blood versus pressure of blood characteristic, according to aspects of the present embodiments. The volume of the blood VB within each of the right and left ventricles 32, 34 may be determined using the volume of the air VA within the drive system 40, 42 (to which it is inversely proportional). The overall volume of the total artificial heart 30 is constant (or approximately constant) so as the volume of air in the drive system 40, 42 increases, the volume of blood in the right and left ventricles 32, 34 must decrease. Similarly, as the volume of air in the drive system 40, 42 decreases, the volume of blood in the right and left ventricles 32, 34 must increase. The pressure of the blood PB may be determined based on the volume of blood VB in each of the right and left ventricles 32, 34 using an inverse relationship. When the volume of blood in the right and left ventricles 32, 34 is at a maximum value (as marked by a first point “A” in FIGS. 29 and 30) the volume of the air VA within the drive system 40, 42 will be at a minimum value. Similarly, when the volume of blood in the right and left ventricles 32, 34 is at a minimum value (as marked by a second point “B” in FIGS. 29 and 30) the volume of the air VA within the drive system 40, 42 will be at a maximum value. As such, curve 218 (in FIG. 30) corresponds to a resting condition when the total artificial heart 30 is pumping the minimum amount of blood, while curve 216 of FIG. 30 corresponds to an active (or maximum cardiac output) condition when the total artificial heart 30 is pumping the maximum amount of blood.

FIG. 31 illustrates a schematic of a volume of air VA versus pressure of air PA characteristic, according to aspects of the present embodiments. In the embodiment of FIG. 31, if the VA versus PA characteristic shifts from an initial curve 220 to a new curve 222 such that for a given volume, there is additional air pressure, it could be an indication that something has changed in the total artificial heart system 60. For example, because the amount of air and the volume ranges of the system do not change over time, a shift in the VA versus PA characteristic similar to that of FIG. 31 may be an indicator that external pressure is acting on the drive system 40, 42 (for example, due to increased blood pressure acting on the diaphragm 36, 38 and/or follower dome 110), thereby indicating that mitigating action should be taken (for example, the patient should visit the doctor, or have his or her blood pressure checked using a separate system such as a sphygmomanometer).

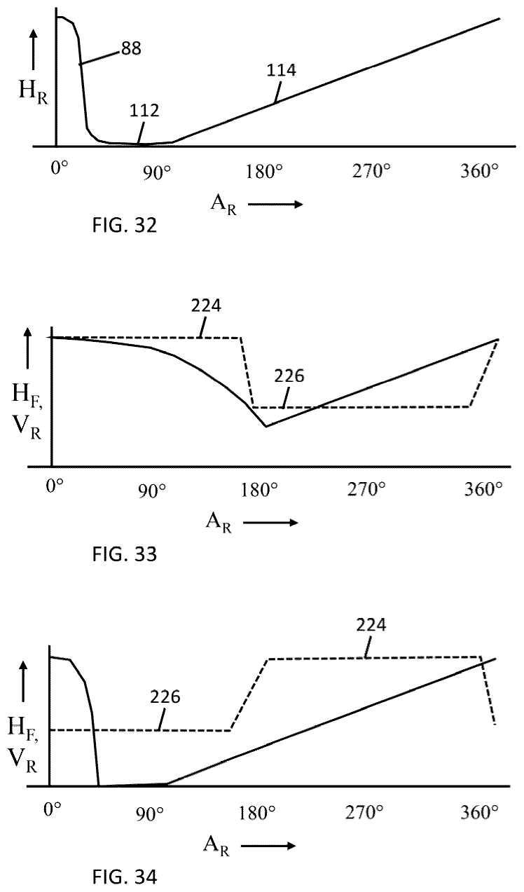

FIG. 32 illustrates a schematic of ramp height HR versus rotor angle AR characteristic, according to aspects of the present embodiments. The ramp height HR varies at any given fixed circumferential location as the rotorcam 64 rotates through various rotor angles AR. For example, at a rotor angle AR of zero (0) degrees, the ramp height HR may be at a maximum. As the rotorcam 64 rotates to ninety (90) degrees such that the drop-off 88 passes under the one or more teeth 72, the ramp height is at a minimum value (for example, at the flat portion 112). As the rotorcam 64 rotates to one hundred and eighty (180) degrees, two-hundred and seventy (270) degrees, and eventually to three-hundred and sixty (360) degrees, the ramp height HR steadily increases on the inclined portion 114, eventually reaching a maximum height at three-hundred and sixty (360) degrees (or zero (0) degrees).

FIG. 33 illustrates a schematic of both follower height HF and rotor velocity VR versus rotor angle AR characteristics, according to aspects of the present embodiments. The illustration of FIG. 33 assumes a system with the same ramp height HR profile as that of FIG. 32. In the embodiment of FIG. 33, the rotor velocity VR (as represented by the dashed line) is at higher velocity 224 from about zero (0) degrees to about one-hundred and eighty (180) degrees, and at a lower velocity 226 from about one-hundred and eighty (180) degrees to about three-hundred and sixty (360) degrees. The electric motor 140 may control the rotational speed, even within a single rotation, based on the Hall sensor 118, 120 data (i.e., which can be used to determine both rotational speed VR and circumferential location of the rotorcam 64). As the drop-off 88 of the rotorcam 64 passes under the one or more teeth 72, the rotorcam 64 is rotating at the higher velocity 224. As a result, the tooth 72 does not make contact with the ramp 70 until the rotorcam 64 has rotated about one-hundred and eighty (180) degrees. Stated otherwise, the height of the follower HF drops from zero (0) degrees to one-hundred and eighty (180) degrees, but because the rotorcam 64 is spinning so quickly, the cam follower 66 never touches the flat portion 112. Instead, the cam follower 66 first makes contact with the ramp 70 (i.e., after the drop-off 88 passes) at the inclined portion 114. Because the cam follower 66 is not dropping all the way down in the embodiment of FIG. 33, the volume of the right and left ventricles 32, 34 never reaches a maximum value. Accordingly, the embodiment of FIG. 33 corresponds to a resting or minimum cardiac output condition.

FIG. 34 illustrates a schematic of both follower height HF and rotor velocity VR versus rotor angle AR characteristics, according to aspects of the present embodiments. The illustration of FIG. 34 assumes a system with the same ramp height HR profile as that of FIG. 32 (and also FIG. 33). In the embodiment of FIG. 33, the rotor velocity VR (as represented by the dashed line) is at a lower rotational velocity 226 from about zero (0) degrees to about one-hundred and eighty (180) degrees, and at a higher rotational velocity 224 from about one-hundred and eighty (180) degrees to about three-hundred and sixty (360) degrees. As the drop-off 88 of the rotorcam 64 passes under the one or more teeth 72, the rotorcam 64 is rotating at the lower velocity 226. As a result, the tooth 72 drops down and makes contact with the flat portion 112. Stated otherwise, the height of the follower HF drops down much more quickly in the embodiment of FIG. 34, than that of FIG. 33. Because the cam follower 66 is dropping all the way down in the embodiment of FIG. 34, the volume of the right and left ventricles 32, 34 does reach a maximum value. Accordingly, the embodiment of FIG. 34 corresponds to an active or maximum cardiac output condition.

Referring to FIGS. 33 and 34, the area above the follower height HF curve (i.e., the solid curve) is directly proportional to the cardiac output of the total artificial heart 30. As such, the lower the cam follower 66 drops, the higher the cardiac output of the total artificial heart 30 will be. The cam follower 66 will drop down more quickly as the rotational speed between zero (0) degrees and about one-hundred and eighty (180) degrees is decreased. The rotor angle AR may also be described as a circumferential orientation of the rotorcam 64. In the embodiments of both FIGS. 33 and 34, the average rotational speed across the full three-hundred and sixty (360) degree rotation may be identical. However, in FIG. 33, the rotorcam 64 rotates quicker during the first portion of the rotation, resulting in a lower overall cardiac output. By contrast, in FIG. 34, the rotorcam 64 rotates slower during the first portion of the rotation, resulting in a higher overall cardiac output. Stated otherwise, the magnitude of the expansion of the right and/or left artificial ventricle 32, 34 is inversely proportional to the rotational speed of the rotorcam 64. As such, the cardiac output can be adjusted higher and lower by adjusting the rotational speed within each rotation, without changing the overall average rotational speed (i.e., or heartrate).

Each of the operational characteristics illustrated in FIGS. 28-34 may be used (individually and/or in concert with one another) to detect changes in one or more conditions related to the patient's health. For example, if any of the operational characteristics illustrated in FIGS. 28-34 changes over time, an alert may be sent to the patient and/or a clinician indicating that the patient's blood pressure has changed, or that one or more potentially harmful conditions such as hypervolemia, pulmonary hypertension, blood clot, and others may exist.

### Methods of Operation

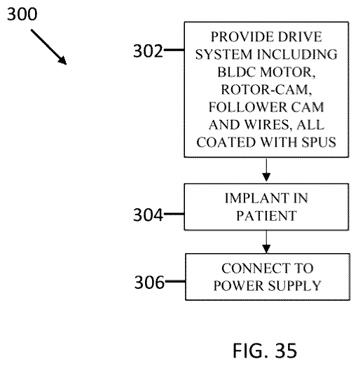

FIG. 35 illustrates a schematic of a method 300 of implanting a total artificial heart 30 in a human body, according to aspects of the present embodiments. At step 302, the method 300 may include providing a system including an electric motor (such as a BLDC motor 140) including a stator assembly 62, a rotorcam 64, a cam follower 66, and wires 48, where at least one of the system components is coated with a segmented polyurethane solution (SPUS). At step 304, the method 300 may include implanting the system into a patient. At step 306, the method 300 may include connecting a power supply to the implanted system.

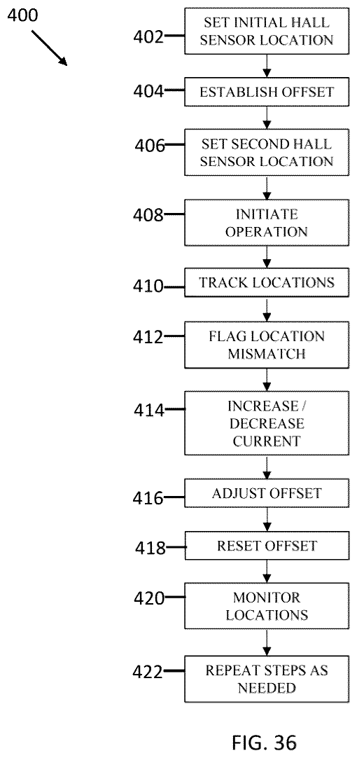

FIG. 36 illustrates a schematic of a method 400 of calibrating a total artificial heart 30, according to aspects of the present embodiments. At step 402, the method 400 may include setting an initial Hall sensor 136 location within the rotorcam 64 or the stator assembly 62 such that the Hall sensor 136 may detect changes in the electromagnetic field associated with each rotation of the rotorcam 64. At step 404, the method 400 may include establishing an offset between a first drive system 40 and a second drive system 42 such that the right and left ventricles 32, 34 may be actuated (or pumped) in an ordered fashion with a contraction of the right ventricle 32 immediately following the contraction of the left ventricle 34 (or vice versa). In other embodiments, the offset may be established by rotationally offsetting the rotorcam 64 within the second drive system 42 from that of the first drive system 40 (i.e., set them at different initial angles) such that even though electricity may be introduced to each drive system simultaneously, the first and second drive systems 40, 42 will be at different portions of the pumping cycle. At step 406, the method 400 may include setting a second Hall sensor 136 location corresponding to the location of the second rotorcam 64 (i.e., the rotorcam 64 of the second drive system 42). At step 408, the method 400 may include placing the total artificial heart 30 in operation. At step 410, the method 400 may include tracking the locations of each of the rotorcams 64 within the respective first and second drive systems 40, 42 such that the offset may be monitored. At step 412, the method may include flagging (or sending an alert indicating) when the initial offset has drifted. At step 414, the method 400 may include slightly increasing or decreasing the electrical current (thereby controlling the rotational speed) to one of the two drive systems 40, 42 (while maintaining the other one at a constant value) thereby adjusting the offset (at step 416) to a new value or resetting the offset (at step 418) back to the initial value. At step 420, the method may include monitoring the locations of each rotorcam 64 (and the angular offset therebetween) to determine if a change in the offset has occurred. At step 422, the method 400 may include repeating any of steps 402-420 as needed, in order to achieve a desired rotorcam 64 offset and/or calibration of the system.

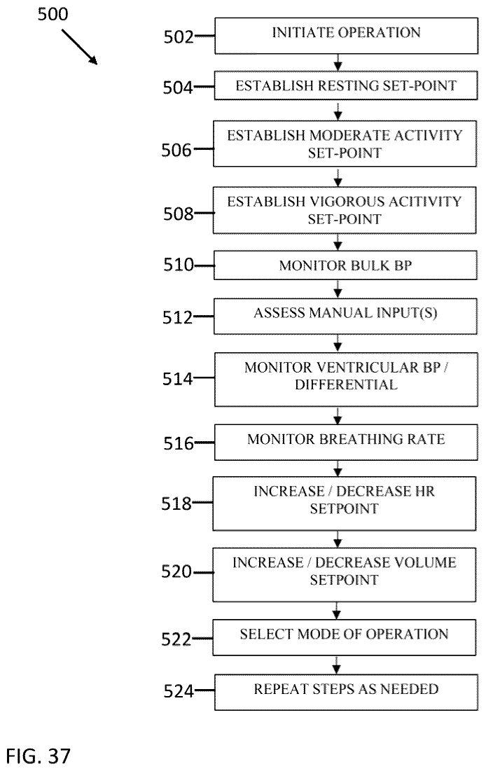

FIG. 37 illustrates a schematic of a method 500 of operating a total artificial heart 30, in accordance with aspects of the present disclosed embodiments. At step 502, the method 500 may include initiating operation of the total artificial heart system 60. At step 504, the method 500 may include establishing a resting set-point (for example a resting heartrate, a resting blood volume, and/or a resting compression ratio of the drive system 40, 42). At step 506, the method 500 may include establishing a moderate activity set-point (i.e., corresponding to the desired cardiac output associated with moderate activity). At step 508, the method 500 may include establishing a vigorous activity set-point (for example setting a heartrate, blood volume, or compression ratio that corresponds to the desired cardiac output for vigorous activity). At step 510, the method 500 may include monitoring a bulk blood pressure (for example, as determined via FIGS. 28-31, or via a separate device such as a sphygmomanometer). At step 512, the method 500 may include assessing manual inputs that the patient or user may have made at the control interface or mobile power supply 54 (i.e., at the heartrate control module 166 or the volume control module 174), for example to increase or decrease the cardiac output (i.e., from one of the setpoints). At step 514, the method 500 may include monitoring the blood pressure within each of the right and left artificial ventricles 32, 34 so as to determine if there is a difference between the two (which may be indicative, for example, of pulmonary hypertension or a blood clot). At step 516, the method 500 may include monitoring the breathing rate via the one or more accelerometer or motion sensors 82 mounted near a patient's waist (i.e., in order to assess if a cardiac output should be increased, for example, to match an elevated patient breathing rate). At step 518, the method 500 may include increasing or decreasing one or more heartrate set-points so as to adjust the cardiac output to a desired level based on a breathing rate, a manual input, a bulk blood pressure, a ventricular blood pressure differential, and/or another factor. At step 520, the method 500 may include increasing or decreasing one or more blood volume set-points so as to adjust the cardiac output to a desired level based on a breathing rate, a manual input, a bulk blood pressure, a ventricular blood pressure differential, and/or another factor. At step 522, the method 500 may include selecting a different mode of operation (for example, manual, resting, moderate exercise, breathing rate matching, vigorous exercise, hybrid control, and/or another control mode). At step 524, the method 500 may include repeating any of steps 502-522 as needed to achieve a desired mode of operation of the total artificial heart system 60.

Referring to FIGS. 35, 36, 37, each of the embodiments of the methods 300, 400, 500 may include steps not illustrated in the respective figures. In addition, in one or more embodiments of each of the methods 300, 400, 500, the steps may be performed in a different order than what is illustrated. In other aspects of the present embodiments, each of the methods 300, 400, 500 may also omit one or more steps. In one or more embodiments, each of the methods 300, 400, 500 may include performing at least one step concurrently with at least one other step.

As the skilled person will now readily appreciate, embodiment structured according to the idea of the invention provide an article of manufacture that contains at least a cam-and follower system, which system includes (a) a monolithic rotorcam configured to operate as both a rotor and a cam of the system and to rotate about a centerline (such monolithic rotorcam comprising at least two angled ramps circumferentially extending around a first axis of the monolithic rotorcam, an operational height of the cam of the system defined by a height of an angled ramp of the at least two angled ramps) and (b) a cam follower that has a second axis of the cam follower and a dome having a center of curvature of the dome on the centerline. In this cam-and-follower system either (i) the at least two angled ramps include an outer ramp (having an outer ramp radius) and at inner ramp (having an inner ramp radius that is smaller than the outer ramp radius) or (ii) the cam follower includes at least two teeth extending from the dome toward the monolithic rotorcam (here, each of the at least two teeth is dimensioned to contact a corresponding angled ramp of the at least two angled ramps when the cam-and-follower system is assembled), or both. In at least one embodiment of the article of manufacture, the cam follower is devoid of constituent components configured to move with respect to one another; and/or the cam follower is monolithic; and/or a spatial profile of a tooth of the at least two teeth is not congruent with a spatial profile of the corresponding angled ramp of the at least two angled ramps. Alternatively or in addition—and substantially in every implementation of the article of manufacture—the at least two teeth may include exactly two teeth that are circumferentially spaced apart by a predetermined angle as viewed along the second axis; and/or each of the outer ramp and the inner ramp of the rotorcam may include a corresponding drop-off and—when the cam follower has exactly two teeth that are circumferentially spaced apart by a chosen angle as viewed along the second axis—a drop-off of the outer ramp and a drop-off of the inner ramp may also be circumferentially spaced apart by the same chosen angle. Alternatively or in addition, and substantially in every implementation of the article of manufacture, at least one of the following conditions may be satisfied: (1) when the cam follower has exactly two teeth that are angularly spaced apart as viewed along the second axis, an angular separation between first and second of the exactly two teeth is about 180 degrees to ensure approximately 50-percent duty cardiac cycle during the use of the article of manufacture in a total artificial heart system implanted in a chest cavity of a patient; and (2) the monolithic rotorcam includes at least three angled ramps circumferentially extending around the first axis (here, the cam follower has at least three teeth respectively corresponding the at least three ramps of the monolithic rotorcam). Alternatively or in addition—and substantially in every implementation of the article of manufacture—each of angled ramps of the monolithic rotorcam may be dimensioned to include a corresponding inclined portion having a first ramp height at a first ends thereof and a second ramp height at a second end thereof, a corresponding drop-off portion sharing the second end with the inclined portion and having a drop-off surface that is substantially transverse to an upper surface of the inclined portion, and a corresponding flat portion disposed between said inclined portion and said drop-off portion and having an upper surface of the flat portion that substantially seamlessly merging with the upper surface of the inclined portion and the drop-off surface (in at least one specific case, a height of the ramp remains substantially equal to the first ramp height along the flat portion and the second ramp height is larger than the first ramp height). In at least some embodiments, a height of each angled ramp of the monolithic rotorcam may be structured to be monotonically changing between ends of an inclined portion of such angled ramp, and—such angled ramp includes a flat portion—the height of such angled ramp along the flat portion may remain substantially constant. (In one specific case, the includes portion may extend circumferentially about the first axis for about 270 degrees and the flat portion may extend circumferentially for about 90 degrees, while a drop-off surface is substantially parallel to the first axis.) In substantially every embodiment of the article of manufacture, the first and second of the at least two angled ramps are preferably radially separated from one another by a radial gap (which radial gap has an outer radius and an inner radius, each of which remains substantially constant as a function of azimuthal angle about the first axis). Substantially in every embodiment of the article of manufacture, the cam follower may be additionally dimensioned to have a shaft monolithically extending from the dome along a radius of a curvature of a convex surface of the dome, while the monolithic rotorcam may include an axial center bore that is dimensioned to accommodate the shaft therethrough. Alternatively or in addition, substantially every embodiments of the article of manufacture may include a stator assembly having a third axis, a base plate substantially perpendicular to the third axis, and a base housing extending from the base plate along the third axis and defining a hollow that is substantially centered at the third axis. (Here, when the cam follower has a shaft monolithically extending from the dome along a radius of a curvature of a convex surface of the dome, an inner perimeter of the hollow may be substantially geometrically matching an outer perimeter of a shaft of the cam follower that extends along the second axis way from an outer surface of the dome. Optionally, the inner perimeter of the hollow may be defined by a polygon, and/or the stator assembly may include multiple groups of wound coil wires disposed about the inner perimeter and configured to be electrically activated sequentially around the third axis. In at least one case, the article of manufacture may be configured to include a drive system having an electric motor. Such electric motor includes (when the rotorcam and the stator assembly and the cam follower are assembled to have the first axis and the second axis and the third axis substantially coincide): the rotorcam sandwiched between the stator assembly and the cam follower such that the shaft passes through a center bore of the rotorcam and is received and accommodated within the hollow of the stator assembly without an ability to rotate about the second axis within the hollow; and a first of the at least two teeth in contact with a ramp surface of one of the inner ramp and the outer ramp and a second of the at least two teeth be in contact with a ramp surface of the other of the inner ramp and an outer ramp. (Optionally, in such a case, the article of manufacture has an axial length defined by an axial separation between a surface of the base plate and a surface of the dome.) In more than one embodiment of the article of manufacture, the cam-and-rotor system may be configured to be implanted in a chest cavity of a patient, and such article may additionally include an artificial ventricle (configured to be implanted in a chest cavity of a patient and coupled to the cam-and-follower system) and be configured (a) to change a magnitude of expansion of the artificial ventricle substantially inversely proportionally to a rotational speed of the rotocam, and/or (b) to change a volume of the artificial ventricle by changing a height of the cam follower with respect to a base on the rotorcam. Embodiments of the invention additionally provide a process that is performed with the use of substantially every implementation of the article of manufacture identified above (that has been implanted in the chest cavity of a patient in contact with a flexible diaphragm of an artificial ventricle that has also been implanted in the chest cavity) and that includes (A) the step rotating a monolithic rotocam, configured to operate as both a rotor and a cam of a cam-and-follower system of said article, about a centerline; and

(B) the step of changing a magnitude of expansion of the artificial ventricle inversely proportionally to a rotations speed of the rotorcam, and/or changing a volume of the artificial ventricle by changing a height of the cam follower with respect to a base on the rotorcam.

The skilled artisan will further appreciate that embodiments of the invention provide an article of manufacture that includes at least a cam-and-follower system, which system includes a monolithic rotorcam (configured to operate as both a rotor and a cam of the system and to rotate about a centerline and having a first axis and containing an angled cam ramp circumferentially extending around the first axis and including at least one contoured cam lobe extending along the first axis, said angled cam ramp having a first edge surface) and a cam follower (having a second axis and a dome and including an angled cam follower ramp circumferentially extending around a perimeter of the dome and including at least one contoured cam follower lobe, such angled cam follower ramp having a second edge surface dimensioned to be substantially congruent with the first end surface). The cam follower of the article of manufacture is configured to be mechanically coupled to the monolithic rotorcam when the cam-and-follower system is assembled such that in an axial separation between the first and second edge surfaces is substantially zero. In at least one specific case, the cam follower is devoid of constituent components configured to move with respect to one another and/or the cam follower is monolithic. In at least one case, a height of each of the angled cam ramp and the angled cam follower ramp is monotonically and uninterruptingly changing around a corresponding circumference. Alternatively or in addition, and substantially in every implementation of the article of manufacture, the cam follower may have a shaft monolithically extending from the dome along a radius of a curvature of a convex surface of the dome, while the monolithic rotorcam includes an axial center bore that is dimensioned to accommodate the shaft therethrough. Substantially in every implementation, the article of manufacture may optionally additionally include a stator assembly having a third axis, a base plate substantially perpendicular to the third axis, and a base housing extending from the base plate along the third axis and defining a hollow that is substantially centered at the third axis (here, when the cam follower has a shaft monolithically extending from the dome along a radius of a curvature of a convex surface of the dome, an inner perimeter of the hollow is preferably but not necessarily substantially geometrically matching an outer perimeter of a shaft of the cam follower that extends along the second axis way from an outer surface of the dome; optionally, the inner perimeter of the hollow may be a polygon, and/or the stator assembly may include multiple groups of wound coil wires disposed about the inner perimeter and configured to be electrically activated sequentially around the third axis). Substantially in every embodiment, the article of manufacture may be configured to include a drive system with an electric motor that includes (when the rotorcam and the stator assembly and the cam follower are assembled to have the first axis and the second axis and the third axis substantially coincide) (i) the rotorcam sandwiched between the stator assembly and the cam follower such that the shaft passes through a center bore of the rotorcam and is received and accommodated within the hollow of the stator assembly without an ability to rotate about the second axis within the hollow. Optionally, the axial length of the article of manufacture is defined by an axial separation between a surface of the base plate and a surface of the dome. Optionally, the cam-and-rotor system may be configured to be implanted in a chest cavity of a patient while the article of manufacture additionally includes an artificial ventricle (configured to be implanted in a chest cavity of a patient and coupled to the cam-and-follower system) and is configured (1) to change a volume of the artificial ventricle substantially inversely proportionally to a rotational speed of the rotocam, and/or (2) to change a volume of the artificial ventricle by changing a separation between the dome of the cam follower and the circular base of the stator assembly.

As used herein, the terms “left” and “right” are interpreted in a frame of reference taken from the patient's perspective, which may be opposite the location in which it appears on the page. For example, in reference to FIG. 2, the left artificial ventricle 34 is located on the right side of the Figure, while the right artificial ventricle 32 is located on the left side of the Figure.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value. As a non-limiting example, two values being “substantially equal” to one another implies that the difference between the two values may be within the range of +/−20% of the value itself, preferably within the +/−10% range of the value itself, more preferably within the range of +/−5% of the value itself, and even more preferably within the range of +/−2% or less of the value itself.

The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes. Other specific examples of the meaning of the terms “substantially”, “about”, and/or “approximately” as applied to different practical situations may have been provided elsewhere in this disclosure.

The expression of the type “element A and/or element B” has the meaning that covers embodiments having element A alone, element B alone, or elements A and B taken together and, as such, is intended to be equivalent to “at least one of element A and element B”.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention. Within this specification, embodiments have been described in a way that enables a clear and concise specification to be written, but it is intended and will be appreciated that embodiments may be variously combined or separated without parting from the scope of the invention. In particular, it will be appreciated that all features described herein are applicable to all aspects of the invention.

When the present disclosure describes features of embodiments of the invention with reference to corresponding drawings (in which like numbers represent the same or similar elements, wherever possible), the depicted structural elements are generally not to scale, and certain components may be enlarged or reduced in size relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, at least for purposes of simplifying the given drawing and discussion, and directing the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this particular detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

What is claimed is:

1. A total artificial heart system comprising:
an artificial ventricle configured to be implanted in a chest cavity of a patient and to be coupled to at least one a blood vessel extending out of or arising from a heart, and a chamber of a heart; and
a drive system having an electric motor that includes a stator assembly, a rotocam coupled to the stator assembly, and a cam follower coupled to the rotorcam,
wherein the drive system is configured to be implanted in a chest cavity of a patient, to be coupled to the artificial ventricle, and configured such that a magnitude of a change of a volume of the artificial ventricle is substantially inversely proportional to a rotational speed of the rotorcam.

2. A system according to claim 1, further comprising a diaphragm configured as a boundary between the artificial ventricle and the drive system,
wherein on a ventricle side, the at least one diaphragm is configured to contact human blood, and
wherein on a side of the drive system, the at least one diaphragm is configured to contact a gas.

3. A system according to claim 1, wherein the drive system is configured to cause the change in size of the artificial ventricle by changing a height of the cam follower.

4. A system according to claim 1, wherein:
the rotorcam is a monolithic rotorcam that is configured to operate as both a rotor and a cam of the system and to rotate about a centerline that that comprises at least two angled ramps circumferentially extending around a first axis of the monolithic rotorcam; and
the cam follower has a second axis of the cam follower and a dome having a center of curvature of the dome on the centerline;
wherein:
(i) the at least two angled ramps comprise:
an outer ramp having an outer ramp radius; and
at inner ramp having an inner ramp radius that is smaller than the outer ramp radius;
and/or
(ii) the cam follower comprises at least two teeth extending from the dome toward the monolithic rotorcam, wherein each of the at least two teeth is dimensioned to contact a corresponding angled ramp of the at least two angled ramps when the cam-and-follower system is assembled.

5. A system according to claim 1, wherein:
the rotorcam is a monolithic rotorcam that is configured to operate as both a rotor and a cam of the system and to rotate about a centerline and that has first axis and comprises an angled cam ramp circumferentially extending around the first axis and including at least one contoured cam lobe extending along the first axis, said angled cam ramp having a first edge surface; and
the cam follower has a second axis and a dome and comprises an angled cam follower ramp circumferentially extending around a perimeter of the dome and including at least one contoured cam follower lobe, said angled cam follower ramp having a second edge surface dimensioned to be substantially congruent with the first end surface.

6. A system according to claim 1, further comprising:
a power supply electrically configured to be placed externally to the chest cavity while coupled to the electric motor that has been implanted to the chest cavity, and to be electrically coupled with said electric motor via an electric member.

7. A system according to claim 6, wherein said power supply comprises a control interface configured to adjust a cardiac output of the total artificial heart system.

8. A system according to claim 1, comprising a right artificial ventricle configured to be coupled to a pulmonary artery and a left artificial ventricle configured to be coupled to an atrium;
the system further comprising:
a first drive system that includes a first electric motor configured to be implanted in the chest cavity and to be coupled to the right artificial ventricle, and
a second drive system that includes a second electric moto configured to be implanted in the chest cavity and to be coupled to the left artificial ventricle.

9. A system according to claim 1, comprising:
multiple artificial ventricles, each configured to be implanted in the chest cavity;
multiple drive systems,
wherein each chosen of the multiple drive systems includes a corresponding electric motor, is configured to be implanted in the chest cavity, and corresponds to and is coupled with one respective of the multiple artificial ventricles to vary a size of said one respective of the multiple artificial ventricles by an amount that is substantially inversely proportional to a rotational speed of a rotorcam of said chosen of the multiple drive systems; and
wherein:
(9A) the corresponding electric motor comprises a brushless direct current (BLDC) motor, and/or
(9B) the corresponding electric motor comprises at least one Hall sensor, and/or
(9C) a plurality of winding groups of the corresponding electric motor comprises at least six winding groups, and/or
(9D) each winding group of said plurality of winding groups comprises one or more longitudinally aligned conductive coil wires, and/or
(9E) an inner stator housing of the corresponding electric motor comprises a hexagonal housing while an outer stator housing of the corresponding electric motor comprises a cylindrical housing, and/or
(9F) a rotor of the corresponding electric motor comprises at least one permanent magnet, and/or
(9G) wherein, when the at least one rotor comprises at least one permanent magnet, the at least one permanent magnet comprises at least one of neodymium iron boron (Nd—Fe—B), iron, cobalt, samarium cobalt (Sm—Co), aluminum, alnico, one or more rare earth elements, bonded Nd—Fe—B, magnetite, ceramic (hard ferrite), ferrite, gadolinium, strontium, barium, and iron (III) oxide.

10. A system according to claim 1, wherein:
(10A) the at least one electric motor comprises:
an inner stator housing,
a plurality of winding groups disposed radially outward of the inner stator housing,
an outer stator housing disposed radially outward of the plurality of winding groups, and
a rotor disposed radially outward of the outer stator housing; and/or
(10B) the stator assembly comprises:
a circular base,
at least one housing longitudinally extending from the circular base, and
a plurality of winding groups disposed radially inward of and/or and radially outward of the at least one housing, and wherein the rotor is disposed radially outward of a housing of the stator.

11. A system according to claim 1, further comprising:
a mobile power supply configured to be operably connected to the artificial ventricle and/or the drive system;
an electronic device that includes a display screen,
a tangible non-transitory storage system containing a computer readable program code disposed thereon to track activity of at least the artificial ventricle and the drive system;
and
a communications module configured to wirelessly communicate at least one data parameter between the electronic device and the mobile power supply.

12. A system according to claim 1, configured to satisfy at least one of the following conditions:
(12A) wherein the computer readable code comprises a series of program steps to display at least one data parameter, that has been transmitted from the mobile power supply to the electronic device, on the display screen;
(12B) wherein the at least one data parameter comprises at least one of a heartrate and a blood pressure of the patient in whom the total artificial heart system has been implanted;
(12C) wherein the at least one data parameter comprises at least one of a charge level of a battery operably connected to the mobile power supply and a remaining life of said battery; and
(12D) wherein the at least one data parameter comprises at least one of a blood volume of a ventricle of the patient carrying the total artificial heart system, an operating mode of the total artificial heart system, and an operating status of the total artificial heart system.

13. A system according to claim 1,
wherein the system further characterized by at least one of the following features:
(13A) the system includes at least one spring coupling the cam follower to the stator assembly, wherein the stator assembly comprises a shaped housing with a first shaped cross-section; wherein the at least one cam follower comprises a shaft with a second shaped-cross section matching the first shaped cross-section of the shaped housing; and
(13B) wherein the at rotorcam includes:
a center bore concentric about a centerline of the rotorcam; and
a ramp radially disposed around the center bore; and
(13C) wherein the rotorcam comprises at least one of neodymium iron boron (Nd—Fe—B), iron, cobalt, samarium cobalt (Sm—Co), aluminum, alnico, bonded Nd—Fe—B, magnetite, ceramic (hard ferrite), ferrite, gadolinium, one or more rare earth elements, strontium, barium, and iron (III) oxide; and
(13D) the system further comprises at least one proximity sensor disposed within at least one of the stator assembly and the cam follower, wherein the at least one proximity sensor is configured to detect a relative position of the cam follower and the stator assembly with respect to one another; and
(13E) the system further comprises at least one pressure sensor disposed on at least one of the stator assembly and the rotorcam; and
(13F) wherein the rotorcam comprises a rotocam bore, the stator assembly comprises a cylindrical housing, and
wherein the cylindrical housing is disposed within the rotorcam bore such that the rotorcam extends circumferentially around, and radially outward of, the cylindrical housing;
and
(13G) wherein the stator assembly comprises a stator assembly cylindrical housing, wherein the rotorcam is disposed about the stator assembly cylindrical housing, and wherein the system is configured to have a rotation of the rotorcam about the stator assembly cylindrical housing to cause a change in a height of the cam follower.

14. A system according to claim 13, wherein, when the system includes the at least one spring and wherein the stator assembly comprises the shaped housing with the first shaped cross-section and wherein the cam follower comprises the shaft with said second shaped-cross section:
each of the first and second shaped cross-sections includes at least one of a triangular cross-section, a square cross-section, an elliptical cross-section, a pentagonal cross-section, a hexagonal cross-section, and a rectangular cross-section.

15. A method comprising:
with the use of the total artificial heart system according to claim 1:
(15A) calibrating said total artificial heart system by at least:
positioning a first Hall sensor and a second Hall sensor respectively at a first initial location and a second initial location defined within the electric motor of the drive system such as to have an initial offset between said first and second initial locations;
initiating operation of the electric motor;
identifying when an offset between time-dependent locations of the first and second Hall sensors has altered from a value of the initial offset; and
varying an electrical current to the electric motor to adjust the offset to a target value;
and/or
(15B) establishing, at a control interface of the total artificial heart, at least one set-point comprising a target heartrate of the total artificial heart system for a given activity mode; and
changing a cardiac output of the total artificial heart system based on at least one data parameter received at the control interface.

16. A method according to claim 15, further comprising:
(16A) when performing said calibrating, monitoring the time-dependent locations of the first and second Hall sensors; and/or
(16B) when using said control interface, assessing at least one manual input, wherein:
(i) the at least one manual input includes altering a heartrate of the total artificial heart system at a heartrate control module coupled to the control interface; and/or
(ii) the at least one manual input includes altering a volume of blood pumped by the total artificial heart system at a volume control module coupled to the control interface.

17. A total artificial heart system comprising:
an artificial ventricle configured to be implanted in a chest cavity of a patient and to be coupled to at least one a blood vessel extending out of or arising from a heart, and a chamber of a heart; and
a drive system having an electric motor that includes a stator assembly, a rotocam coupled to the stator assembly, and a cam follower coupled to the rotorcam,
wherein the drive system is configured to be implanted in a chest cavity of a patient, to be coupled to the artificial ventricle, and configured to change a volume of the artificial ventricle by changing a height of the cam follower.

18. An article of manufacture comprising:
a cam-and-follower system that includes:
a monolithic rotorcam configured to operate as both a rotor and a cam of the system and to rotate about a centerline, the monolithic rotorcam comprising at least two angled ramps circumferentially extending around a first axis of the monolithic rotorcam, an operational height of the cam of the system defined by a height of an angled ramp of the at least two angled ramps; and
a cam follower that has a second axis of the cam follower and a dome having a center of curvature of the dome on the centerline,
wherein:
(18A) the at least two angled ramps comprise:
an outer ramp having an outer ramp radius; and
at inner ramp having an inner ramp radius that is smaller than the outer ramp radius;
and/or
(18B) the cam follower comprises at least two teeth extending from the dome toward the monolithic rotorcam, wherein each of the at least two teeth is dimensioned to contact a corresponding angled ramp of the at least two angled ramps when the cam-and-follower system is assembled.

19. A method comprising:
with the use of an article of manufacture according to claim 18, implanted in a chest cavity of a patient in contact with a flexible diaphragm of an artificial ventricle that has been implanted in the chest cavity:
(19A) rotating a monolithic rotocam, configured to operate as both a rotor and a cam of a cam-and-follower system of said article, about a centerline; and
(19B) changing a magnitude of expansion of the artificial ventricle inversely proportionally to a rotations speed of the rotorcam, and/or
changing a volume of the artificial ventricle by changing a height of the cam follower with respect to a base on the rotorcam.

20. An article of manufacture comprising:
a cam and follower system that includes:
a monolithic rotorcam configured to operate as both a rotor and a cam of the system and to rotate about a centerline, the monolithic rotorcam having a first axis and comprising an angled cam ramp circumferentially extending around the first axis and including at least one contoured cam lobe extending along the first axis, said angled cam ramp having a first edge surface; and
a cam follower having a second axis and a dome and comprising an angled cam follower ramp circumferentially extending around a perimeter of the dome and including at least one contoured cam follower lobe, said angled cam follower ramp having a second edge surface dimensioned to be substantially congruent with the first end surface,
wherein the cam follower is configured to be mechanically coupled to the monolithic rotorcam when the cam-and-follower system is assembled such that in an axial separation between the first and second edge surfaces is substantially zero.

21. An article of manufacture of claim 20, wherein the cam follower is devoid of constituent components configured to move with respect to one another; and/or
wherein the cam follower is monolithic.
